(19) United States
(12) Patent Application Publication
(10) Pub. No.: US 2023/0255943 A1
(43) Pub. Date: Aug. 17, 2023

(54) MMP-9 AND MMP-12 INHIBITION FOR TREATING SPINAL CORD INJURY OR RELATED INJURY TO NEUROLOGICAL TISSUE

(71) Applicant: The University of Birmingham, Birmingham (GB)

(72) Inventors: Swarnaltha Balasubramanian, Birmingham (GB); Zubair Ahmed, Birmingham (GB)

(21) Appl. No.: 18/012,561

(22) PCT Filed: Jun. 25, 2021
(86) PCT No.: PCT/GB2021/051616

§ 371 Date: Dec. 22, 2022

Related U.S. Application Data
(60) Provisional application No. 63/044,496, filed on Jun. 26, 2020.

(51) Int. Cl. A61K 31/426; A61K 31/4439; A61P 25/00

(52) U.S. Cl. CPC A61K 31/426; A61K 31/4439; A61P 25/00

(57) ABSTRACT

The use of a single compound or a combination of compounds which selectively inhibit both matrix metalloproteinases MMP-9 and MMP-12 in treating spinal cord injury (SCI), the secondary effects of SCI and related injury to neurological tissue are described, in particular the suppression of SCI-induced oedema, treatment of neuropathic pain, the prevention of functional decline following SCI and methods of administration.

### FIELD

The present invention concerns certain compounds that inhibit matrix metalloproteinases and are useful in treating spinal cord injury (SCI), particularly the secondary effects associated with SCI, and related injury to neurological tissue.

### BACKGROUND

SCI is a devastating condition that causes significant disability and mortality. Globally, SCI incidence ranges from 15 to 40 cases per million with more than 12,000 and 1,000 new cases in the US and UK each year, respectively. The most common causes of traumatic SCI are motor vehicle accidents, falls, sports-related injuries and interpersonal violence. The clinical outcomes of SCI depend on the severity and location of the lesion and may include partial or complete loss of sensory and/or motor function below the level of the injury. Current treatments of SCI, such as Lyrica (pregabalin), offer only symptomatic relief to patients and do not address the underlying mechanisms that cause neuropathic pain (NP), inflammation, loss of sensory/locomotor function or blood-spinal cord barrier (BSCB) breakdown. A fully restorative therapeutic for SCI would address protection of neurons and glia from further damage, neutralization of inhibitory molecules, inhibition of the glial scar, replacement of lost neurons, axonal regeneration and appropriate synapse formation.

However, the SCI field lacks neuroprotective and neuro-regenerative therapies that have been specifically approved by the FDA or EMEA. Methylprednisolone (Medrol) continues to be used off-label since the effectiveness of the drug is uncertain and the high doses required for beneficial effects pose significant harmful side-effects. Several promising treatments for SCI are currently in clinical trials but most are in early phase studies and, currently, Lyrica is the only FDA approved drug that is used to control SCI-induced neuralgia and NP.

The initial mechanical trauma after spinal cord injury (SCI) leads to disruption of the blood-spinal cord barrier (BSCB), oedema, neuronal death, axonal damage and demyelination, followed by a cascade of secondary events that expand the inflammatory reaction around the epicentre of the original injury.

Matrix metalloproteases (MMPs) are zinc-dependent endopeptidases that target extracellular matrix (ECM) proteins, chemokines, cytokines and cell surface receptors. To date 23 MMPs have been identified in humans, of which MMP-2, MMP-9 and MMP-12 are implicated in SCI, and robustly increase immediately after SCI, with the extent of expression being directly proportional to the severity of the injury. Excessive MMP activity contributes to disruption of the BSCB, oedema, excitotoxicity, mitochondrial apoptosis, inflammation, astrogliosis, NP and loss of function. Therefore, blocking excessive MMP activity after SCI is a useful strategy that will prevent further damage to axonal tracts in the spinal cord, since oedema is correlated with clinical neurological deficits.

MMP-9 expression peaks within one day after SCI and is detected in glia, macrophages, neutrophils and vascular element in the SCI at 24 hours after injury. Excessive activity of MMP-9 in the acute phase of SCI contributes to disruption of the BSCB, oedema, excitotoxicity, influx of leukocytes, mitochondrial dysfunction, apoptosis, demyelination of neurons, increase in inflammatory responses and astrogliosis. In addition, MMP-9 overexpression regulates early-phase neuropathic pain through cleavage of the proinflammatory cytokine, interleukin-1 (IL-1) and microglial activation in a rat sciatic nerve ligation mode.

MMP-12 is an elastinolytic protease that is mainly expressed by macrophages and increases 189-fold in a compression SCI model, peaking at 5 days after injury. MMP-12 expression after SCI is detrimental and contributes to the inflammatory response, acute oedema and development of the secondary injury response. Therefore, blocking oedema, a key pathological event after SCI, is a useful strategy that will prevent further damage to axonal tracts in the spinal cord, since oedema is correlated with neurological deficits.

Separate genetic knockout of MMP-9 and MMP-12 has demonstrated improved functional recovery, reduced BSCB breakdown and reduced neuropathic and inflammatory pain in animal studies, providing evidence that blocking MMP-9 or MMP-12 may have beneficial neuroprotective effects. The adverse role for MMP-12 in intracerebral haemorrhage (ICH) and SCI has been reported with the authors proposing that MMP-12 may be key to outcome from ICH injury in humans (respectively, J. E. Wells, J. Biernaskie, A. Szymanska, P. H. Larsen, V. W. Yong, D. Corbett, Matrix metalloproteinase (MMP)-12 expression has a negative impact on sensorimotor function following intracerebral haemorrhage in mice. Eur J Neurosci 21, 187-196 (2005) and J. E. Wells, T. K. Rice, R. K. Nuttall, D. R. Edwards, H. Zekki, S. Rivest, V. W. Yong, An adverse role for matrix metalloproteinase 12 after spinal cord injury in mice. J Neurosci 23, 10107-10115 (2003)).

Inhibitors of MMP's, including broad spectrum inhibitors, have been developed and tested in animal models including GM6001 (a broad spectrum MMP inhibitor), Inhibitor I (a MMP-9 selective inhibitor), SB-3CT, Lipitor, Fluoxetine and Sulforaphane. See the following references:

Y. Kawasaki, Z. Z. Xu, X. Wang, J. Y. Park, Z. Y. Zhuang, P. H. Tan, Y. J. Gao, K. Roy, G. Corfas, E. H. Lo, R. R. Ji, Distinct roles of matrix metalloproteases in the early- and late-phase development of neuropathic pain. Nat Med 14, 331-336 (2008).

H. Kobayashi, S. Chattopadhyay, K. Kato, J. Dolkas, S. Kikuchi, R. R. Myers, V. I. Shubayev, MMPs initiate Schwann cell-mediated MBP degradation and mechanical nociception after nerve damage. Mol Cell Neurosci 39, 619-627 (2008).

F. Yu, H. Kamada, K. Niizuma, H. Endo, P. H. Chan, Induction of mmp-9 expression and endothelial injury by oxidative stress after spinal cord injury. J Neurotrauma 25, 184-195 (2008).

C. K. Wada, J. H. Holms, M. L. Curtin, Y. Dai, A. S. Florjancic, R. B. Garland, Y. Guo, H. R. Heyman, J. R. Stacey, D. H. Steinman, D. H. Albert, J. J. Bouska, I. N. Elmore, C. L. Goodfellow, P. A. Marcotte, P. Tapang, D. W. Morgan, M. R. Michaelides, S. K. Davidsen, Phenoxyphenyl sulfone N-formylhydroxylamines (retrohydroxamates) as potent, selective, orally bioavailable matrix metalloproteinase inhibitors. J Med Chem 45, 219-232 (2002).

H. Zhang, M. Chang, C. N. Hansen, D. M. Basso, L. J. Noble-Haeusslein, Role of matrix metalloproteinases and therapeutic benefits of their inhibition in spinal cord injury. Neurotherapeutics 8, 206-220 (2011).

Although these inhibitors showed promise in early animal models they have never been tested in SCI in humans. This is probably due to fears of possible detrimental musculoskeletal (MSK) side-effects of MMP inhibitors that have been observed in other conditions due to the length of use (at least 6 weeks), especially with broad-spectrum MMP inhibitors (J. T. Peterson, Matrix metalloproteinase inhibitor development and the remodeling of drug discovery. Heart Fail Rev 9, 63-79 (2004)).

US Patent Application US 2003/0139332 A1 (Noble et al) relates to MMP, particularly MMP-9, inhibitors in SCI & associated neurological damage.

Zhang et al (Neurotherapeutics, Vol. 8, 206-220, April 2011) relates to MMPs (particularly MMP-9) & their inhibitors in SCI. Zhang et al mentions that MMP-9 assists wound healing. Hence, inhibiting MMP-9 should not help wound healing and could lead to scarring. Zhang also states that use of broad-spectrum MMP inhibitors in the more chronically injured cord should be approached with caution.

Wells et al (The Journal of Neuroscience, Nov. 5, 2003-23(31), 10107-10115) relates to MMP-12, its high upregulation in SCI and the potential use of MMP inhibition in SCI.

The present disclosure relates to the combined selective inhibition of the activity or expression of both matrix metalloproteinase MMP-9 (gelatinase-B) and metalloelastase MMP-12 together after SCI or related injury to neurological tissue.

AZD1236 is a potent, reversible and specific inhibitor of human MMP-9 and MMP-12, with 10 to 15-fold selectivity to MMP-2 and MMP-13 and >350-fold selectivity to other members of the enzyme family. The IC₅₀ of AZD1236 has been measured as 4.5 nM and 6.1 nM for MMP-9 and MMP-12, respectively. AZD1236 has been used clinically in chronic obstructive pulmonary disease (COPD) in randomized control 6-week trials, with patients receiving 75 mg twice a day.

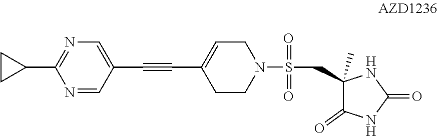 [SMILES: C[C@]1(CS(=O)(=O)N2CC=C(C#Cc3cnc(C4CC4)nc3)CC2)NC(=O)NC1=O]

The preparation of AZD1236 is described in WO 2006/004532 (see e.g. Example 1, p. 20). The full chemical name for AZD1236 is (5S)-5-({[4-(2-cyclopropylprimidin-5-yl)ethynyl]-3,6-dihydropyridin-1(2F)-yl]sulfonyl}methyl]-5-methylimidazolidine-2,4-dione.

AZD3342 is another potent inhibitor of human MMP-9 and MMP-12 with an IC₅₀ of 10 nM and 6 nM (5.9 nM) measured for MMP-9 and MMP-12, respectively.

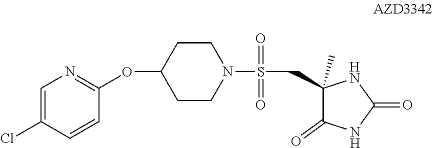 [SMILES: C[C@]1(CS(=O)(=O)N2CCC(Oc3ccc(Cl)cn3)CC2)NC(=O)NC1=O]

The preparation of AZD3342 is described in WO 2002/074767 (see e.g. p. 65, lines 15-27; p. 120, lines 23-29) and novel crystalline forms are disclosed in WO 2007/106022. The full chemical name for AZD3342 is (5S)-5-[4-(5-chloro-pyridin-2-yloxy)-piperidine-1-sulfonylmethyl]-5-methylimidazolidine-2,4-dione.

### SUMMARY

Herein it is shown that certain compounds that selectively inhibit both MMP-9 and MMP-12 are useful in the treatment of SCI or related injury to neurological tissue. In particular, it is shown that certain compounds that selectively inhibit both matrix metalloproteinases MMP-9 and MMP-12 block SCI-induced oedema, suppress inflammatory pain (including neuropathic pain), reduce BSCB breakdown, reduce scarring and prevent sensory and locomotor functional decline following SCI and related injury to neurological tissue. Certain compounds that selectively inhibit both MMP-9 and MMP-12 also promote axon regeneration and sparing of axons above and below a SCI lesion site.

Thus, in one embodiment there is provided a compound or a combination of compounds for use in treating spinal cord injury (SCI) or related injury to neurological tissue (such as traumatic brain injury (TBI)) or for use in treating the secondary effects associated with SCI or related injury to neurological tissue (such as TBI), comprising selectively inhibiting the activity or expression of both MMP-9 (gelatinase-B) and metalloelastase MMP-12 after such SCI or related injury to neurological tissue.

In a further embodiment there is provided a compound or combination of compounds for use in treating the secondary effects associated with SCI or related injury to neurological tissue, comprising selectively inhibiting the activity or expression of both MMP-9 (gelatinase-B) and metalloelastase MMP-12 after such SCI or related injury to neurological tissue.

In a further embodiment there is provided a compound or combination of compounds for use as described herein wherein a single selective MMP-9 and MMP-12 inhibitor or a combination of a selective MMP-9 inhibitor or inhibitors and a separate selective MMP-12 inhibitor or inhibitors is used.

In a further embodiment there is provided a combination of compounds for use as described herein wherein a separate selective MMP-9 inhibitor or inhibitors and a separate selective MMP-12 inhibitor or inhibitors are used.

In a further embodiment there is provided a combination of compounds for use as described herein wherein a combination of a separate selective MMP-9 and a separate selective MMP-12 inhibitor or inhibitors is used (preferably a single MMP-9 inhibitor and a single MMP-12 inhibitor, i.e. a two-compound combination).

In a further embodiment there is provided a single compound for use as described herein wherein a single compound which is both a selective MMP-9 inhibitor and MMP-12 inhibitor is used.

In a further embodiment the MMP-12 inhibitor or inhibitors have higher activity than the MMP-9 inhibitor or inhibitors used. In further embodiments, the MMP-12 inhibitory activity is 10, 100 or 1,000 times higher than that of the MMP-9 inhibitory activity.

In a further embodiment the MMP-9 inhibitor or inhibitors have higher activity than the MMP-12 inhibitor or inhibitors used. In further embodiments, the MMP-9 inhibitory activity is 10, 100 or 1,000 times higher than that of the MMP-12 inhibitory activity.

By “selectively inhibiting the activity or expression of both MMP-9 and MMP-12” it is meant that the activity or expression of both MMP-9 and MMP-12 is inhibited and to a higher extent than inhibition of other MMP's, particularly MMP-2. The inhibition of both MMP-9 and MMP-12 is simultaneous when both MMP-9 and MMP-12 are simultaneously inhibited, although this may be more sequential in nature if levels of MMP-9 and MMP-12 fluctuate during a course of treatment and either MMP-9 or MMP-12 is inhibited more initially and then the other is inhibited more significantly later in the treatment. However, although MMP-9 and MMP-12 levels may fluctuate during treatment both should always be present to some extent (i.e. there will always be at least some simultaneous inhibition of both MMP-9 and MMP-12). Thus, in the uses/treatments described herein there should always be a selective inhibitor or inhibitors of both MMP-9 and MMP-12 simultaneously present.

Thus, there is provided a compound or combination of compounds for use in treating spinal cord injury (SCI) or related injury to neurological tissue or for use in treating the secondary effects associated with SCI or related injury to neurological tissue, comprising selectively (and simultaneously) inhibiting to a higher extent than inhibition of other MMP's, particularly MMP-2, the activity or expression of both matrix metalloproteinase MMP-9 (gelatinase-B) and metalloelastase MMP-12 after such SCI or related injury to neurological tissue.

In one embodiment there is selectively inhibition of both the activity and expression of both MMP-9 and MMP-12. In a further embodiment there is selective inhibition of the activity of both MMP-9 and MMP-12.

In one embodiment the inhibition of MMP-9 and MMP-12 is at least 10 times more selective than inhibition of any other MMP. In another embodiment, the inhibition of MMP-9 and MMP-12 is at least 50 or 100 times more selective than inhibition of any other MMP. In a further embodiment, the inhibition of MMP-9 and MMP-12 is at least 300 times more selective than inhibition of any other MMP.

MMP-9 and MMP-12 may be simultaneously inhibited by a single compound possessing both MMP-9 and MMP-12 inhibitory activity or by a combination of separate compounds, one being a selective MMP-9 inhibitor and the other a selective MMP-12 inhibitor. The administration of separate compounds may be in a fixed dose combination composition comprising both compounds or by separate, sequential or simultaneous administration of compositions comprising the individual compounds, provided that both compounds are administered so that inhibition of both MMP-9 and MMP-12 occurs at the same time.

In one embodiment, by “selectively inhibiting the activity or expression of both MMP-9 and MMP-12”, it is also meant that the compound or combination of compounds used have approximately equal activity against both MMP-9 and MMP-12. For example, the IC₅₀ of AZD1236 has been measured as 4.5 nM and 6.1 nM for MMP-9 and MMP-12, respectively; the IC₅₀ of AZD3342 has been measured as 10 nM and 6 nM for MMP-9 and MMP-12, respectively.

In one embodiment, there is provided a single compound for use as described herein, wherein the single selective MMP-9 and MMP-12 inhibitor compound has an IC₅₀ against both MMP-9 and MMP-12 in the range of 1 nM to 50 nM or, in another embodiment, in the range 1 nM to 100 nM.

In a further embodiment, there is provided a single compound for use as described herein, wherein the single selective MMP-9 and MMP-12 inhibitor compound has an IC₅₀ of greater than 100 nM or, in another embodiment, greater than 200 nM, against MMP-2.

In a further embodiment, there is provided a single compound for use as described herein, wherein the single selective MMP-9 and MMP-12 inhibitor compound has an IC₅₀ against both MMP-9 and MMP-12 in the range of 1 nM to 50 nM (or, in another embodiment, in the range 1 nM to 100 nM) and an IC₅₀ of greater than 200 nM against MMP-2.

In a further embodiment, the single selective MMP-9 and MMP-12 inhibitor compound for use as described herein is AZD1236 or AZD3342, or a pharmaceutically acceptable salt of these.

In a further embodiment, the single selective MMP-9 and MMP-12 inhibitor compound for use as described herein is AZD1236, or a pharmaceutically acceptable salt thereof.

In further embodiments, the MMP-12 inhibitory activity is 10, 100 or 1,000 times higher than that of the MMP-9 inhibitory activity.

In further embodiments, the MMP-9 inhibitory activity is 10, 100 or 1,000 times higher than that of the MMP-12 inhibitory activity.

In another embodiment, there is provided a compound or a combination of compounds for use in treating neuropathic pain, comprising selectively inhibiting the activity or expression of both MMP-9 (gelatinase-B) and metalloelastase MMP-12. Neuropathic pain is pain caused by damage or disease affecting the somatosensory nervous system. In one embodiment, there is provided AZD1236, or a pharmaceutically acceptable salt thereof, for use in treating neuropathic pain.

### DESCRIPTION OF FIGURES

This disclosure is illustrated and supported by the accompanying Figures.

FIG. 1. MMP-9 and MMP-12 levels and their enzymatic activity increase acutely after DC injury. (A) MMP-9 mRNA increases to a maximum by 1d after injury whilst (B) MMP-12 peaks at 5d after injury. (C) MMP-9 protein levels also peak 1 day after injury. (D) MMP-12 protein levels also peak at 5 days after injury. (E) MMP-9 activity is high at 1 day and peaks by 3 days after injury. RFU=relative fluorescence units. (F) MMP-12 activity peak at 5 days after injury. RFU=relative fluorescence units. Data are expressed as means±SEM. n=6 mice/group, 2 independent experiments, total n=12 mice/group. P=0.0001, one-way ANOVA with Dunnett's post hoc test.

FIG. 2. AZD1236 significantly suppresses MMP-9 and MMP-12 activity. (A) and (B) MMP-9 and MMP-12 activity is suppressed by oral delivery of AZD1236 in both serum and CSF, respectively. (C) and (D) MMP-9 and MMP-12 activity is suppressed by intrathecal delivery of AZD1236 in both serum and CSF, respectively. (E) Summary table to show % suppression of MMP-9 and MMP-12 activity by AZD1236 after oral and intrathecal delivery in serum and CSF, respectively. (F) Optimal doses of AZD1236 also significantly suppress MMP-9 and MMP-12 activity in spinal cord homogenates (RFU=relative fluorescence units). (G) In situ zymography shows that the high levels of gelatinase enzyme activity (green; arrowheads) after DC injury is suppressed after oral and intrathecal delivery of optimal doses of AZD1236 in spinal cord sections. Sections are counterstained with GFAP (red) to mark astrocytes in red. #=lesion site. Data are expressed as means±SEM. n=6 mice/group, 2 independent experiments, total n=12 mice/group. ***=P=0.0001, one-way ANOVA with Dunnett's post hoc test. Scale bars in (G)=200 μm.

FIG. 3. Spinal cord water content. (A) The mean water content (oedema) in the spinal cord rises and peaks at 3 days after DC injury and thereafter declines, compared to sham-treated control levels. AZD1236 at (B) 200 mg/kg and at (C) 5 mg/kg attenuates DC injury-induced rises in spinal cord water content at 3d after oral and intrathecal delivery, respectively, immediately after injury. (D) Inhibition of both MMP-9 and MMP-12 is required to ablate SCI-induced edema. Note: MMP408 and Inhibitor I were used at doses above when used singly, however, when combined doses were halved (i.e. 100 mg/kg=50 mg/kg MMP408/50 mg/kg Inhibitor I, 200 mg/kg=100 mg/kg MMP408/100 mg/kg Inhibitor I, 300 mg/kg=150 mg/kg MMP408/150 mg/kg Inhibitor I). Data are expressed as means±SEM. n=6 mice/group, 2 independent experiments, total n=12 mice/group. **=P=0.01; ***=P=0.0001, one-way ANOVA with Dunnett's post hoc test. AZD1236 was delivered by oral gavage immediately after injury.

FIG. 4. Comparison of MMP inhibitors and their ability to suppress water content in the spinal cord at 3 days after DC injury. Dorsal column (DC) injury induces and increase in water content which is only partially suppressed by different inhibitors of MMPs. (A) Mean water content of the spinal cord after treatment with GM6001 (broad spectrum MMP inhibitor; inhibits MMP-1, MMP-2, MMP-3, MMP-8 and MMP-9). * P=0.05, one-way ANOVA with Dunnett's post hoc test. (B) Mean water content of the spinal cord after treatment with SB-3CT (selective MMP-2 inhibitor). (C) Mean water content of the spinal cord after treatment with MMP-9 Inhibitor I. *=P=0.05, one-way ANOVA with Dunnett's post hoc test. (D) Mean water content of the spinal cord after treatment with SD2590 (inhibits MMP-2, MMP-3, MMP-8, MMP-9, MMP-13 and MMP-14). *=P=0.05, one-way ANOVA with Dunnett's post hoc test. (E) Mean water content of the spinal cord after treatment with ND378 (inhibits MMP-2). (F) Mean water content of the spinal cord after treatment with Minocycline (no effect on MMP activity). (G) Mean water content of the spinal cord after treatment with Riluzole (GABA uptake inhibitor). *=P=0.05, one-way ANOVA with Dunnett's post hoc test. (H) Mean water content of the spinal cord after treatment with Glibenclamide (Sur1-regulated NCCa-ATP). *=P=0.05, one-way ANOVA with Dunnett's post hoc test. (I) Mean water content of the spinal cord after treatment with Melatonin (aquaporin-4). *=P=0.05, one-way ANOVA with Dunnett's post hoc test. (J) Compared to all MMP inhibitors used in this study, AZD1236 was far superior, almost completely ablating DC injury-induced rises and suppressing these levels back to sham-treated control levels. ***=P=0.00012, one-way ANOVA with Dunnett's post hoc test. Data are expressed as means±SEM. n=6 mice/group, 2 independent experiments, total n=12 mice/group. *=P=0.05, one-way ANOVA with Dunnett's post hoc test. AZD1236 was delivered by oral gavage immediately after injury.

FIG. 5. Inhibition of MMP-9 and MMP-12 suppresses proinflammatory pain markers after DC injury. (A) and (B) Inhibition of MMP-9 and MMP-12 by AZD1236 attenuates mRNA levels of proinflammatory pain markers interleukin-1β (IL-1β), tumor necrosis factor-α (TNF-α) and interleukin-6 (IL-6) in the DC injury model after oral and intrathecal delivery respectively. Data are expressed as means±SEM. n=6 mice/group, 2 independent experiments, total n=12 mice/group. ***=P<0.0001, one-way ANOVA with Dunnett's post hoc test.

FIG. 6. AZD1236 suppresses spinal cord water content, proinflammatory pain markers, MMP activity and improvements in pain behaviours in the clip-compression (CC) model of SCI. AZD1236 either by oral or intrathecal (it) delivery attenuates: (A) injury-induced rise in spinal cord water content, (B) relative expression of proinflammatory pain markers, (C) MMP-9 and MMP-12 activity, and oral delivery improves responses to (D) tactile, (E) thermal and (F) cold allodynia. Note: AZD1236 confers significant reductions in pain behaviours compared to currently used neuropathic pain medications, pregabalin (30 mg/kg) and gabapentin (100 mg/kg) (preoptimized concentrations determined in preliminary tests in the CC model prior to full experiments). Data are expressed as means±SEM. n=6 mice/group, 2 independent experiments, total n=12 mice/group. *=P=0.04 **=P=0.01; ***=P=0.0001, one-way ANOVA with Dunnett's post hoc test. AZD1236 was delivered by oral gavage immediately after injury.

FIG. 7. AZD1236 is more effective at reducing proinflammatory pain markers after CC injury than other currently used pain medications. AZD1236 significantly attenuates proinflammatory pain markers IL-13, TNF-α and IL-6 after CC injury compared to pre-optimised pregabalin and gabapentin, which were much less effective. Data are expressed as means±SEM. n=6 mice/group, 2 independent experiments, total n=12 mice/group. *=P=0.04 **=P=0.01; ***=P=0.0001, one-way ANOVA with Dunnett's post hoc test. All drugs were delivered by oral gavage immediately after injury.

FIG. 8. Oral delivery of AZD1236 improves electrophysiological, locomotor and sensory outcomes after DC injury. (A) Representative superimposed CAP traces after oral delivery of AZD1236. (B) CAP amplitudes and (C) CAP area both show significant improvements after treatment with AZD1236. (D) Ladder crossing (locomotor function) and (E) mean tape sensing/removal times (sensory function) both show significant improvements after treatment with AZD1236. #=P<0.00147, linear mixed models; ##=P<0.00114, generalised linear mixed models; ***=P<0.0001, ANOVA. Data are expressed as means±SEM. n=6 mice/group, 3 independent experiments, total n=18 mice/group.

FIG. 9. Intrathecal delivery of AZD1236 improves electrophysiological, locomotor and sensory outcomes after DC injury. (A) Representative superimposed CAP traces after intrathecal delivery of AZD1236. (B) CAP amplitudes and (C) CAP area both show significant improvements after treatment with AZD1236. (D) Ladder crossing (locomotor function) and (E) mean tape sensing/removal times (sensory function) both show significant improvements after treatment with AZD1236. #=P<0.0015, linear mixed models; ##=P<0.0011, generalised linear mixed models; ***=P<0.0001, ANOVA. Data are expressed as means±SEM. n=6 mice/group, 3 independent experiments, total n=18 mice/group.

FIG. 10. Inhibition of MMP-9 and MMP-12 using AZD1236 promotes DC axon regeneration and axon sparing above and below the lesion in mouse DC injury. (A) Cholera toxin B (CTB) retrogradely labelled axons to regenerating/sprouting within the DC lesion site and growing into the rostral cord of animals treated with DC+AZD126 while no evidence of CTB labelled axons were present in the lesion site or beyond in DC+Vehicle-treated controls. Scale bars=200 μm. (B) Significantly greater proportions of CTB labelled axons were quantified rostra/caudal to the lesion site in DC-AZD1236-treated animals compared to DC+Vehicle-treated controls. (C) Immunohistochemistry to detect Neurofilament (NF) 200⁺ fibres in cross sections of the spinal cord from DC+Vehicle and DC+AZD1236-treated animals, above (T9) and below (T7) the lesion site. Scale bars=200 μm (D) Quantification of the number of NF200 pixels above and below the lesion site to demonstrate sparing of axons in DC+AZD1236-treated animals compared to DC+Vehicle-treated controls. Data are expressed as means±SEM. n=6 mice/group, 3 independent experiments, total n=18 mice/group. ***P=0.0001, one-way ANOVA with Dunnett's post hoc test. AZD1236 was delivered by oral gavage immediately after injury.

FIG. 11. AZD1236 promotes significantly more DC axon regeneration than other MMP inhibitors FIG. 12. Twenty-four hour delayed treatment with AZD1236 is as beneficial as immediate treatment. (A) Data to show that AZD1236 significantly suppressed SCI induced spinal cord water content. ***=P=0.0001, one-way ANOVA with Dunnett's post hoc test. (B) Expression of proinflammatory pain markers are significantly suppressed. ***=P=0.0001, one-way ANOVA with Dunnett's post hoc test. (C) MMP-9 and MMP-12 activity is also suppressed by AZD1236. ***=P=0.0001, one-way ANOVA with Dunnett's post hoc test. (D) Representative Spike 2 processed CAP traces after delayed treatment with AZD1236. Note: At the end of recording, dorsal hemisection of the cord resulted in obliteration of CAP taces demonstrating technical success of the experiment. (E) Significant improvements in (E) CAP amplitudes and (F) CAP areas were observed after treatment with AZD1236. ***=P=0.0001, one-way ANOVA with Dunnett's post hoc test. (G) Ladder crossing and (H) tape sensing and removal performance were improved by AZD1236 treatment. Data are expressed as means±SEM. ##=P<0.0012, generalized linear mixed model. #=P<0.0011, linear mixed models. n=6 mice/group, 2 independent experiments, total n=12 mice/group.

FIG. 13. Twenty-four hour delayed treatment with AZD1236 promotes similiar proportions of axon regeneration as immediate treatment. (A) CTB⁺ axons stopped at the lesion site (#) in DC+Vehicle-treated mice whereas, signficant proportions of CTB⁺ lablled axons were observed regenerating through the lesion site and entering the rostral cord in animals treated with AZD1236.C=caudal, R=Rostral. Scale bars=200 μm. (B) Quantification of the proportion of CTB⁺ regenerating axons shows similar proportions of axons as immediate treatment. ***=P=0.0001, one-way ANOVA with Dunnett's post hoc test. n=6 mice/group, 2 independent repeats (total n=12 mice/group).

FIG. 14. Oral delivery of AZD1236 attenuates blood spinal cord barrier (BSCB) breakdown and scar tissue at the lesion site. (A) Inhibition of MMP-9 and MMP-12 suppressed albumin extravasation as detected by albumin immunoreactivity at lesion site (#) and (B) quantification demonstrated suppressed BSCB breakdown at 3d after DC injury and treatment with AZD1236 compared to vehicle-treated animals. (C) Laminin immunoreactivity (scar tissue) at the lesion site (#) at 4 weeks after DC injury and treatment showed significantly suppressed scar tissues in AZD1236-treated animals compared to vehicle-treated groups. (D) Quantification of the number of laminin immunoreactive pixels demonstrated significantly attenuated levels of laminin scar tissues at the lesion site. (E) Semaphorin-3A (Sema-3A) immunoreactivity and (F) quantification to show that AZD1236 reduces Sema-3A deposition in the injury site (#) when delivered immediately after injury or within 24 hr. (G)CS-56 immunoreactivity and (H) quantification to show that AZD1236 reduces CS-56 deposition in the injury site when delivered immediately after injury or within 24 hr. (1) CD11b, CD68 and GFAP immunoreacitvity in saggital sections from DC+Vehicle and DC+AZD1236-treated animals, respectively. Scale bars in E, G and I=100 μm, in inset i and inset ii in (I)=200 μm. (J-L) Quantification of the number of CD11 b, CD68 and GFAP⁺ immunoreactive pixels demsonstrated significantly suppressed levels in DC+AZD1236 treated animals (immediate and after 24 hr delay). Data are expressed as means±SEM. AU=arbitrary units. n=6 mice/group, 2 independent experiments, total n=12 mice/group. ***P=0.0001, one-way ANOVA with Dunnett's post hoc test. AZD1236 was delivered by oral gavage immediately or at 24 h after injury. Scale bars in (A) and (C)=100 μm.

FIG. 15. Common CSF biomarkers of SCI are also attenuated by AZD1236. (A) S100P, (B) NSE, (C) GFAP, (D) pNF-H and (E) NF-L are all marginally reduced in comparison by Melatonin but significantly attenuated attenuated by AZD1236. n=12 mice/group. ***=P=0.0001, one-way ANOVA with Dunnett's post hoc test.

FIG. 16. AZD1236 attenuates proinflammatory cytokine release from LPS-stimulated microglia but does not affect macrophage migration in vitro. (A) TNF-α, (B) IL-1β and (C) IL-6 production by LPS-stimulated primary microglia is inhibited by AZD1236. In comparison, other MMP inhibitors such as GM6001, SD2590 and MMP Inhibitor I only marginally attenuate TNF-α, IL-1β and IL-6 levels. (D) Macrophage migration is not affected by AZD1236, GM6001, Sd2590 or MMP9 Inhibitor I but positive controls, MCP-1 and PMA, significantly increase migration index in all macrophage populations. n=3 wells/treatment, 3 independent repeats (total n=9 wells/treatment). **=P<0.001; ***=P<0.0001, one-way ANOVA with Dunnett's post hoc test.

FIG. 17. Inhibition of MMP-9 and MMP-12 using AZD3342 is also effective in rat models of DC injury. (A) MMP-9 mRNA levels in the rat also peak at 1 day after DC injury. (B) MMP-12 mRNA levels in the rat also peak at 5 days after DC injury. (C) Spinal cord water content (oedema) is ablated in rats at 3 days after inhibition of MMP-9 and MMP-12. As a comparison, Melatonin only had a marginal effect. (D) AZD3342 significantly suppresses MMP-9 and MMP-12 activity. (E) AZD3342 significantly suppressed the expression of proinflammatory pain cytokines, with Melatonin having a small effect. (F) Representative Spike 2 processed CAP traces at 6 weeks after treatment with AZD3342 and Melatonin. AZD3342 restored a significant CAP wave after DC injury. (G) AZD3342 improved ladder crossing performance (locomotor function) over 6 weeks. (H) AZD3342 improved tape sensing and removal performance times (sensory function) over 6 weeks compared to vehicle or melatonin treated rats. Data are expressed as means±SEM. n=6 mice/group, 2 independent experiments, total n=12 mice/group. ***P=0.0001, one-way ANOVA with Dunnett's post hoc test. #=P<0.0011, linear mixed models; ##=P<0.0011, generalized linear mixed models. AZD3342 was delivered by oral gavage immediately after injury.

FIG. 18. Return of MMP-9 and MMP-12 activity at the lesion site to normal DC-injury-induced levels by 4-5 days after the last oral dose of AZD1236. (A) MMP-9 activity returns to injury-induced levels by day 7, taking 4 days to return to these levels after withdrawal of AZD1236. (B) MMP-12 activity returns to injury-induced levels by day 8, taking 5 days to return to these after after withdrawal of AZD1236. AZD1236 was delivered by oral gavage immediately after injury.

FIG. 19. MMP-2 activity after oral and intrathecal AZD1236 administration. There is no change in MMP-2 activity after oral and intrathecal AZD1236 administration, indicating that the effects are specific to MMP-9 and MMP-12 inhibition. Data are expressed as means±SEM. n=6 mice/group, 2 independent experiments, total n=12 mice/group.

FIG. 20. Water content in the spinal cord as a measure of injury-induced oedema. Dorsal column (DC) injury induces an increase in water content which is completely suppressed by both AZD1236 and AZD3342 (specific inhibitors of MMP-9 and MMP-12).

FIG. 21. Water content after suppression of aquaporin-4. Aquaporin-4 relocalisation inhibitors (trifluoperazine (TFP), protein kinase inhibitor (PKAi) and TGN-020 suppress SCI-induced oedema. The protein kinase C inhibitor had no effect on SCI-induced oedema.

### DETAILED DESCRIPTION

Herein it is shown that inhibiting both matrix metalloproteinases MMP-9 and MMP-12 is useful in the treatment of SCI or related injury to neurological tissue, or in the treatment of the secondary effects associated with SCI or related injury to neurological tissue.

In some embodiments this can be achieved by using a combination of separate compounds with respective MMP-9 and MMP-12 inhibitory activity. In other embodiments, a single compound with both MMP-9 and MMP-12 inhibitory activity can be used.

AZD1236, a specific MMP-9 and MMP-12 inhibitor was used and its effects on oedema, BSCB breakdown, NP, scar formation and functional and behavioural recovery determined after both oral and intrathecal delivery. It was found that AZD1236 inhibits MMP-9 and MMP-12 in spinal cord tissue, serum and cerebrospinal fluid and suppresses SCI-induced oedema, reduces inflammatory pain markers and NP responses (mechanical, thermal and cold allodynia), improves electrophysiological responses across the SCI site, improves locomotor and sensory function and reduces BSCB breakdown and scaring at the lesion site, helping to preserve longer-term function. Furthermore, it was found that inhibition of MMP-9 and MMP-12 by AZD1236 supresses microglial activation and macrophage infiltration at the lesion site and reduces scar-tissue-derived axon growth inhibitory molecules (e.g., Sema-3A and CS-56). All of these effects contribute to an improved environment for axons to regenerate and this promotes sparing of axons above and below the lesion site.

The results disclosed herein also demonstrate that AZD3342 suppresses SCI-induced oedema and that the specific MMP-12 inhibitor, MMP408 in combination with the MMP-9 Inhibitor, Inhibitor I also suppresses SCI-induced oedema.

Herein, it has been demonstrated that the reduction in SCI-induced oedema is reliant on the combination of MMP-9 and MMP-12 being suppressed since suppression of either of these individually provides a suboptimal effect. Clinical outcomes following SCI are greatly affected by spinal cord oedema and if left unchecked can lead to further damage and death.

It has also been found that MMP-12 was expressed at much higher levels than MMP-9 and its expression peaked at 5 days after SCI, suggesting that suppression of MMP-12 in particular has positive roles in reducing BSCB breakdown and greater recovery of function after SCI.

Advantageously, using MMP-9 and MMP-12 inhibitors (for example, AZD1236, AZD3342, or a specific MMP-9 inhibitor and a specific MMP-12 inhibitor) to suppress oedema provides a non-surgical way to reduce swelling; thus, preventing any further complications and/or damage to the trauma site from surgery.

As shown in the Examples, it has also been found that suppression of both MMP-9 and MMP-12 activity is beneficial in reducing BSCB breakdown, thereby suppressing SCI-induced oedema and improved functional recovery. The outcomes after SCI are greatly affected by spinal cord oedema. These outcomes can be long lasting and pose a significant challenge to neurosurgeons. In addition, acute inhibition of BSCB disruption probably protects the spinal cord from the influx of inflammatory cells and blocks extravasation of molecules such as plasma, reducing the toxic effect of inflammatory cells, glutamate and glycine that can be toxic at high concentrations.

As shown in the Examples, MMP 9 and 12 inhibition by AZD1236 also reduced neutrophil infiltration after SCI and decreased white matter damage, implicating neutrophils in impaired locomotor recovery. Neutrophils damage brain tissues by generating reactive oxygen species and proteases, including MMPs. It was also observed that significant suppression of macrophage and microglial activation in AZD1236-treated animals occurred in and around the lesion site. In addition, the Examples show the suppression of LPS-activated in vitro release of proinflammatory and neuropathic pain-related cytokines from primary microglia by AZD1236. Furthermore, the Examples show that AZD1236 had no effects on macrophage migration in vitro, while in-vivo a reduced number of macrophages in the lesion site in AZD1236 treated animals was observed. The reduced number of macrophages in the lesion site in AZD1236 treated animals is thought to be connected to the reduced BSCB. Overall, all of these effects of AZD1236 contribute in the reduction in overall damage to the spinal cord and subsequent impairments in sensory and motor function.

The results disclosed herein demonstrate reduced BSCB breakdown by albumin labelling, which is normally extravasated into the spinal cord parenchyma due to BSCB breakdown. Reduced scarring at the lesion site was also promoted by AZD1236 as well as suppressed astrocyte activation as measured by GFAP labelling, suggesting enhanced protection of spinal cord tissues and its normal architecture. Sema-3A and CSPG are both ECM molecules that are present at the SCI site and are potent axon growth inhibitors. Pharmacological inhibition of Sema-3A and enzymatic degradation of CSPGs result in significant axon regeneration after SCI and hence reduction of these molecules by AZD1236 probably accounts for the increased DC axon regeneration that was observed.

The results herein disclose that suppression of MMP-9 and MMP-12 by AZD1236 promoted CNS axon regeneration/sprouting after DC injury such that AZD1236-treated animals contained significantly enhanced regenerating axons in the lesion site, which extended for long distances rostrally. To compliment this, there was a significant increase in spared fibres, both above and below the lesion site and probably also contributed to the enhanced functional recovery observed after AZD1236 treatment. Enhanced sparing could be due to the attenuated tissue damage, fewer infiltrating cells and reduced activation of microglia in the lesion sited observed after AZD1236 treatment. The enhanced axon regeneration could also be direct or indirect. For example, acute activation of MMP-9 and MMP-12 are both implicated in disruption of the BSCB (Wang X, Jung J, Asahi M, Chwang W, Russo L, Moskowitz M A, et al. Effects of matrix metalloproteinase-9 gene knock-out on morphological and motor outcomes after traumatic brain injury. J Neurosci 2000; 20(18): 7037-42; Noble L J, Donovan F, Igarashi T, Goussev S, Werb Z. Matrix metalloproteinases limit functional recovery after spinal cord injury by modulation of early vascular events. J Neurosci 2002; 22(17): 7526-35), but MMP-9^{-/-} and MMP-12^{-/-} mice display enhanced behavioral recovery after SCI probably due to increased axonal regeneration/plasticity as well as reduced BSCB breakdown (Wells J E, Rice T K, Nuttall R K, Edwards D R, Zekki H, Rivest S, et al. An adverse role for matrix metalloproteinase 12 after spinal cord injury in mice. J Neurosci 2003b; 23(31): 10107-15). In addition, MMP-9^{-/-} mice exhibited a diminished complexity of the glial scar as well as reduced deposition of chondroitin sulphate proteoglycans and NG2 in the subacute stages after SCI (Jones L L, Yamaguchi Y, Stallcup W B, Tuszynski M H. NG2 is a major chondroitin sulfate proteoglycan produced after spinal cord injury and is expressed by macrophages and oligodendrocyte progenitors. J Neurosci 2002; 22(7): 2792-803; Hsu J Y, Bourguignon L Y, Adams C M, Peyrollier K, Zhang H, Fandel T, et aL. Matrix metalloproteinase-9 facilitates glial scar formation in the injured spinal cord. J Neurosci 2008; 28(50): 13467-77; Andries L, Van Hove I, Moons L, De Groef L. Matrix Metalloproteinases During Axonal Regeneration, a Multifactorial Role from Start to Finish. Mol Neurobiol 2017; 54(3): 2114-25), similar to our observations with AZD1236.

MMP-9 and MMP-12 therefore have multiple functions and experimental evidence suggests complex modulatory roles during various aspects of axonal regeneration in the injured CNS (Andries L, Van Hove I, Moons L, De Groef L. Matrix Metalloproteinases During Axonal Regeneration, a Multifactorial Role from Start to Finish. Mol Neurobiol 2017; 54(3): 2114-25). For example, they contribute to early phase phagocytosis and glial scarring responses whilst they facilitate degradation of myelin-derived inhibitors, activation of growth factors, axonal navigation, axonal elongation, growth cone motility, synaptogenesis and reinnervation and eventual remyelination (Gijbels K, Proost P, Masure S, Carton H, Billiau A, Opdenakker G. Gelatinase B is present in the cerebrospinal fluid during experimental autoimmune encephalomyelitis and cleaves myelin basic protein. J Neurosci Res 1993; 36(4): 432-40; Proost P, Van Damme J, Opdenakker G. Leukocyte gelatinase B cleavage releases encephalitogens from human myelin basic protein. Biochem Biophys Res Commun 1993; 192(3): 1175-81; Andries L, Van Hove I, Moons L, De Groef L. Matrix Metalloproteinases During Axonal Regeneration, a Multifactorial Role from Start to Finish. Mol Neurobiol 2017; 54(3): 2114-25). It is herein disclosed that attenuating the early rise of MMP-9 and MMP-12 is beneficial after SCI. As the activity of MMP-9 and MMP-12 returns to normal 5 days after withdrawal of AZD1236 treatment, it is expected that MMP-9 and MMP-12 can then partake in normal healing responses after injury (Andries L, Van Hove I, Moons L, De Groef L. Matrix Metalloproteinases During Axonal Regeneration, a Multifactorial Role from Start to Finish. Mol Neurobiol 2017; 54(3): 2114-25).

The results disclosed herein demonstrate that AZD1236 suppresses proinflammatory pain markers and reduces tactile, thermal and cold allodynia, all hallmarks of neuropathic pain (NP). This data supports the utility of AZD1236 in limiting SCI-induced NP, most likely through the reduction in edema. However, it is also likely that suppression of macrophage and microglial activity in the spinal cord also contributed to suppression of NP, since infiltration of macrophages, activation of microglia and BSCB barrier breakdown correlate with NP (Zhao H, Alam A, Chen Q, M A E, Pal A, Eguchi S, et aL. The role of microglia in the pathobiology of neuropathic pain development: what do we know? Br J Anaesth 2017; 118(4): 504-16; Honjoh K, Nakajima H, Hirai T, Watanabe S, Matsumine A. Relationship of Inflammatory Cytokines From M1-Type Microglia/Macrophages at the Injured Site and Lumbar Enlargement With Neuropathic Pain After Spinal Cord Injury in the CCL21 Knockout (plt) Mouse. Front Cell Neurosci 2019; 13: 525; Takeura N, Nakajima H, Watanabe S, Honjoh K, Takahashi A, Matsumine A. Role of macrophages and activated microglia in neuropathic pain associated with chronic progressive spinal cord compression. Sci Rep 2019; 9(1): 15656).

Therefore, AZD1236's property to reduce macrophage and microglial activation, BSCB breakdown, as well as neutralizing potentially toxic MMP-9 and MMP-12 probably all contribute to limiting infiltrating cells and thus reduce proinflammatory cytokine production. All of these factors together with suppressed edema all contribute to reduced NP in AZD1236-treated animals. NP is a common secondary complication after SCI and is estimated to be prevalent in 61% of reported cases. Post injury NP presents at or below the level of injury and results in reduced quality of life, depression and sleep disturbances. NP is refractory to current pharmacotherapies, of which Lyrica showed the most promise in a randomized controlled clinical trial (Siddall P J, McClelland J M, Rutkowski S B, Cousins M J. A longitudinal study of the prevalence and characteristics of pain in the first 5 years following spinal cord injury. Pain 2003; 103(3): 249-57; Cardenas D D, Nieshoff E C, Suda K, Goto S, Sanin L, Kaneko T, et aL. A randomized trial of pregabalin in patients with neuropathic pain due to spinal cord injury. Neurology 2013; 80(6): 533-9). The potential mechanisms for NP development after SCI have been described in detail elsewhere in the literature but involve peripheral, spinal cord and brain mechanisms (Siddall P J. Management of neuropathic pain following spinal cord injury: now and in the future. Spinal Cord 2009; 47(5): 352-9; Finnerup N B, Baastrup C. Spinal cord injury pain: mechanisms and management. Curr Pain Headache Rep 2012; 16(3): 207-16; D'Angelo R, Morreale A, Donadio V, Boriani S, Maraldi N, Plazzi G, et aL. Neuropathic pain following spinal cord injury: what we know about mechanisms, assessment and management. Eur Rev Med Pharmacol Sci 2013; 17(23): 3257-61).

The results herein disclosed suggests that there are several surprising advantages of AZD1236 over other MMP inhibitors which warrants its use in SCI. For example, (1), AZD1236 was superior in suppressing oedema compared to other tool and clinical MMP inhibitors; (2) AZD1236 was superior in suppressing proinflammatory pain markers and reducing neuropathic pain behaviors compared to other FDA approved neuropathic pain medications (e.g. pregabalin and gabapentin) (3), AZD1236 caused similar changes in all of the parameters measured in our SCI models when delivered either immediately or 24 h after SCI; (4), Short-term delivery of AZD1236 (3 days) significantly reduces the possibility of musculoskeletal side-effects that have been observed with MMP inhibitors when used for long periods of time; (5), AZD1236 promoted axon regeneration and functional recovery; (6) AZD1236 suppresses scar tissue deposition at the lesion site; (6) AZD1236 reduces infiltration of blood borne cells into the CNS; and (7) AZD1236 promotes sensory and locomotor recovery, making it unique in that it fulfils many aspects of a restorative therapy for SCI.

The results disclosed herein demonstrate that short-term inhibition of MMP-9 and MMP-12 profoundly limits the extent of secondary damage to the spinal cord, thus representing a potential approach to target the primary pathophysiology as a first-line treatment of SCI.

Furthermore, the results herein disclose AZD1236, through specific inhibition of MMP-9 and -12, being the first experimental therapy, which targets all four aspects of SCI pathophysiology. AZD1236 reduces SCI-induced oedema, suppresses proinflammatory markers of pain and improves the responsiveness of animals to a variety of NP sources, improves locomotor and sensory function and reduces BSCB breakdown and scar tissue formation at the lesion site. AZD1236 treatment also led to suppressed macrophage, microglia and astrocyte activation, promoted DC axon regeneration through the lesions site and enhanced sparing of axons above and below the lesion site. All of these beneficial effects of AZD1236 treatment contributed to the overall improvements in sensory and locomotor function observed in the study herein disclosed

In addition, intrathecal delivery of AZD1236 required 1/40^{th} of the oral dose to cause the same beneficial effects after SCI. These results suggest that inhibiting MMP-9 and MMP-12 using AZD1236, either by oral or intrathecal delivery, is a promising therapeutic for the treatment of SCI.

The use of lower doses and a much more targeted route than oral administration may also limit potential side-effects. Advantageously, intrathecal doses can result in levels of the compound (or compounds) reaching a steady level in a shorter time frame than if the compound (or compounds) were administered orally.

Furthermore, surprisingly, the effects observed are apparent after administration for only a short duration, for example for just the first 3 days after SCI. In addition, the benefits are also observed if treatment is initiated up to 24 hours after the SCI. Advantageously, this gives a working window for healthcare professionals to treat the patient post SCI without reducing any benefits associated with treatment, i.e., in certain scenarios there may potentially be a delay in initiating treating for a patient, for example, the time it may take for emergency services to reach a patient after a SCI.

The results disclosed herein are believed to be due to the selective inhibition of MMP-9 and MMP-12, which are up-regulated (along with other MMP's) in SCI. Current symptomatic treatments for SCI (e.g. Lyrica) target NP and are highly addictive. Other MMP inhibitors (e.g. in COPD & cancer treatment) have known side effects (e.g. musculoskeletal side effects) so would not be considered attractive candidates. Using broad-spectrum MMP inhibitors or MMP-2 inhibitors even over longer durations, e.g. over 2 weeks, would not provide the effects seen herein with AZD1236.

The selective inhibition of both MMP-9 and MMP-12 in SCI has not been used before and the rapid and significant effects on oedema and NP are surprising. The effects on oedema in particular could help prevent further injury that is usually associated with swelling and so avoid further surgical (decompression) intervention. In addition, as cannabinoid receptors are not targeted, addiction problems are avoided.

Particular compounds for use as described herein for selectively inhibiting the activity or expression of both matrix metalloproteinase MMP-9 (gelatinase-B) and metalloelastase MMP-12 are compounds, or pharmaceutically acceptable salts of these, which are described in WO 2002/074767, WO 2007/106022 & WO 2006/004532. Also included are tautomers, physical forms, polymorphs, solvates, hydrates of such compounds as well as enantiomers, diastereomers and mixtures & racemates of such compounds.

Particular compounds for use as described herein are AZD1236 and AZD3342 or a pharmaceutically acceptable salt of these. Other particular compounds for use as described herein are the specific MMP-12 inhibitor, MMP408 (see Li, W., et al. 2009. J. Med. Chem. 52, 1799) and the MMP-9 Inhibitor, Inhibitor I (see Levin, J. I., et al. 2001. Bioorg. Med. Chem. Lett. 11, 2189).

Furthermore, the effects on oedema indicate that short-term inhibition of aquaporin channels (AQP4) by compounds (such as TFP, PKAi and TGN-020) may also rapidly suppress oedema in SCI and provide similar effects to AZD1236. Combination treatments may thus also be possible and could allow significantly lower doses of each agent to be used. This short-term (one week) effect with AQP4 inhibitors could help avoid long-term side effects (such as water poisoning).

In the CNS, aquaporin-4 (AQP4), a major water channel protein, regulates BSCB permeability during spinal cord oedema. SCI causes changes in the expression of AQP4 and thus the water content in the spinal cord. AQP4 also regulates swelling in astrocytes which plays a major role in cytotoxic oedema after acute SCI. AQP4 null mice displayed reduced SCI-induced oedema and improved functional recovery in the acute phase after SCI. Pharmacological inhibitors of oedema such as melatonin and surgical interventions such as myelotomy have also reduced SCI-induced oedema by inhibiting AQP4.

### Pharmaceutical Compositions and Doses

According to a further aspect there is provided a pharmaceutical composition which comprises a compound or combination of compounds which selectively inhibit the activity or expression of both matrix metalloproteinase MMP-9 (gelatinase-B) and metalloelastase MMP-12, or a pharmaceutically acceptable salt, hydrate or solvate thereof, in association with a pharmaceutically acceptable diluent or carrier.

The compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral or intrathecal administration (for example as a sterile aqueous or oily solution for intravenous, subcutaneous, intraperitoneal or intramuscular dosing or as a suppository for rectal dosing). In a preferred embodiment, the composition is a tablet.

The compositions may be obtained by conventional procedures using conventional pharmaceutical excipients, well known in the art. Thus, compositions intended for oral use may contain, for example, one or more colouring, sweetening, flavouring and/or preservative agents.

An effective amount of a compound or combination of compounds which selectively inhibit the activity or expression of both matrix metalloproteinase MMP-9 (gelatinase-B) and metalloelastase MMP-12 for use in the treatment of spinal cord injury (SCI) or related injury to neurological tissue, or for use in treating the secondary effects associated with SCI or related injury to neurological tissue, is an amount sufficient to treat or prevent a condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.0 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes will naturally vary according to the nature and severity of the conditions, the age and sex of the animal or patient and the route of administration, according to well-known principles of medicine.

In such therapeutic or prophylactic purposes it will generally be administered so that a daily dose in the range, for example, 0.1 mg/kg to 250 mg/kg (more suitably 0.1 mg/kg to 50 mg/kg) body weight is received, given if required in divided doses. In general lower doses will be administered when a parenteral or intrathecal route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

In one embodiment, the active ingredient is administered once daily or twice daily.

In one embodiment, the active ingredient is administered twice daily. In one embodiment, the first daily dose is administered in the morning, and the second daily dose in the evening.

In one embodiment, AZD1236, or a pharmaceutically acceptable salt thereof, is dosed twice daily.

In one embodiment, AZD1236, or a pharmaceutically acceptable salt thereof, is orally dosed at 50 to 200 mg twice daily, such as about 100 mg twice daily, more conveniently about 75 mg twice daily. In one embodiment, AZD1236, or a pharmaceutically acceptable salt thereof, is intrathecally dosed at 1 to 5 mg twice daily, conveniently, 1 to 2.5 mg twice daily, such as about 1.9 mg twice daily.

In one embodiment, the compound or combination of compounds which selectively inhibit the activity or expression of both matrix metalloproteinase MMP-9 (gelatinase-B) and metalloelastase MMP-12 are administered for a duration of up to 3 days, such as a duration of up to 1 day or up to 2 days. Conveniently, the compound or combination of compounds which selectively inhibit the activity or expression of both matrix metalloproteinase MMP-9 (gelatinase-B) and metalloelastase MMP-12 are administered for a duration of 1 day, 2 days or 3 days, conveniently 3 days. Advantageously, it has been found that normal MMP-9 and MMP-12 activity returns 4-5 days after the last dose of the compound or combination of compounds, i.e. well before the healing phase in the central nervous system. Long term inhibition of MMP-9 may lead to detrimental effects due to its role in wound healing but short term inhibition of MMP-9 and MMP-12 has been found to control the initial excessive activation of these enzymes after SCI.

In one embodiment, the first dose of the compound or combination of compounds which selectively inhibit the activity or expression of both matrix metalloproteinase MMP-9 (gelatinase-B) and metalloelastase MMP-12 is administered in the period of up to 24 hours after SCI or related injury to neurological tissue. Advantageously, it has been demonstrated that the beneficial effects due to administering the compounds or combinations of compounds which selectively inhibit the activity or expression of both MMP-9 and MMP-12 are similar if the compound or combination of compounds are administered immediately after SCI or related injury to neurological tissue or if administered at 24 hours after injury.

In one embodiment, the first dose of the compound or combination of compounds which selectively inhibit the activity or expression of both matrix metalloproteinase MMP-9 (gelatinase-B) and metalloelastase MMP-12 is administered in the period of up to 24 hours after SCI or related injury to neurological tissue, and subsequent doses of the compound or combination of compounds are administered for a duration of 1 day, 2 days or 3 days thereafter, conveniently 3 days thereafter. For example, if the first dose is given at 24 hours post injury, the subsequent doses will be administered in the 1 to 3-day period afterwards.

In one embodiment, the first dose of AZD1236, or a pharmaceutically acceptable salt thereof, is administered in the period of up to 24 hours after SCI or related injury to neurological tissue, and subsequent doses of the compound or combination of compound are administrated for a duration of 3 days thereafter.

In one embodiment, the first dose of AZD1236, or a pharmaceutically acceptable salt thereof, is administered in the period of up to 24 hours after SCI or related injury to neurological tissue, and subsequent doses of the compound or combination of compound are administrated for a duration of 3 days thereafter, wherein AZD1236 is dosed twice daily, for example orally dosed twice daily.

In one embodiment, the first dose of AZD1236, or a pharmaceutically acceptable salt thereof, is administered in the period of up to 24 hours after SCI or related injury to neurological tissue, and subsequent doses of the compound or combination of compound are administrated for a duration of 3 days thereafter, wherein AZD1236 is orally dosed at 50 to 200 mg twice daily, such as about 100 mg twice daily, more conveniently about 75 mg twice daily.

In one embodiment, the first dose of AZD1236, or a pharmaceutically acceptable salt thereof, is administered in the period of up to 24 hours after SCI or related injury to neurological tissue, and subsequent doses of the compound or combination of compound are administrated for a duration of 3 days thereafter, wherein AZD1236 is intrathecally dosed at 1 to 5 mg twice daily, conveniently 1 to 2.5 mg twice daily, such as about 1.9 mg twice daily.

In one embodiment, the compound or combination of compounds is administered with food. In another embodiment, the compound or combination of compounds is administered without food.

### Therapeutic Uses and Applications

In one embodiment there is provided a compound or combination of compounds which selectively inhibit the activity or expression of both matrix metalloproteinase MMP-9 (gelatinase-B) and metalloelastase MMP-12, for use in treating spinal cord injury (SCI) or related injury to neurological tissue or for use in treating the secondary effects associated with SCI or related injury to neurological tissue. By SCI is meant any injury which causes trauma and leads to disruption of the blood-spinal cord barrier (BSCB), oedema, neuronal death, axonal damage and demyelination.

By related injury to neurological tissue is meant any injury which causes trauma and leads to disruption of neurological tissue, oedema, neuronal death, axonal damage and demyelination. In one embodiment the related injury to neurological tissue is traumatic brain injury (TBI). TBI is a form of acquired brain injury, which occurs when a sudden trauma causes damage to the brain. TBI can result when the head suddenly and violently hits an object, or when an object pierces the skull and enters brain tissue.

In another embodiment the compound or combination of compounds for use in treating the secondary effects associated with SCI or related injury to neurological tissue are used for treating SCI-induced oedema or neuropathic pain (NP).

Suitably, the compound or combination of compounds are used for treating neuropathic pain. Suitably, the compound or combination of compounds suppresses the proinflammatory pain markers IL-1p, TNF-α and/or II-6 after SCI or related injury to neurological tissue. Suitably, the compound or combination of compounds suppresses the proinflammatory pain markers IL-1β, TNF-α and/or II-6 after SCI or related injury to neurological tissue by at least 10%, 20%, 30%, 40%, 50%, 60% 70%, 80%, or 90%. Conveniently, the compound is AZD1236, or a pharmaceutically acceptable salt thereof. It is to be understood that suppression of the proinflammatory pain markers is relative to the levels in the subject prior to treatment with the compound or combination of compounds.

Suitably, the compound or combination of compounds are used for treating SCI-induced oedema. Suitably, the compound or combinations of compounds reduces SCI-induced oedema by at least 10, 20, 30, 40, 50, 60, 70, 80, or 90%. Conveniently, the compound is AZD1236, or a pharmaceutically acceptable salt thereof. It is to be understood that the reduction is relative to the oedema in the subject prior to treatment with the compound or combination of compounds.

Other secondary effects associated with SCI or related injury to neurological tissue include reduction in BSCB breakdown and scarring at the SCI lesion site, preservation of longer-term (neurological) function, wound treatment, the prevention of scarring (including combination treatment with decorin), and/or promotion of axon regeneration.

Suitably, the compound or combination of compounds are used for treating scarring in the CNS. Suitably, the compound or combinations of compounds reduces deposition of scar-related Sema-3A and CS-56 by at least 10, 20, 30, 40, 50, 60, 70, 80, or 90%. Conveniently, the compound is AZD1236, or a pharmaceutically acceptable salt thereof. It is to be understood that the reduction is relative to the scarring in the subject prior to treatment with the compound or combination of compounds.

Suitably, the compound or combination of compounds are used for suppressing infiltration of blood-borne cells into the CNS. Suitably, the compound or combinations of compounds reduces blood-borne cells such as monocytes, neutrophils, granulocytes, macrophages and/or natural killer cells by at least 10, 20, 30, 40, 50, 60, 70, 80, or 90%. Conveniently, the compound is AZD1236, or a pharmaceutically acceptable salt thereof. It is to be understood that the reduction is relative to the infiltration of blood-borne cells in the subject prior to treatment with the compound or combination of compounds.

Examples of other conditions which may be treated by use of a compound or combination of compounds which selectively inhibiting the activity or expression of both matrix metalloproteinase MMP-9 (gelatinase-B) and metalloelastase MMP-12 include, but are not limited to, high blood pressure or hypertension, Alzheimer's disease, Huntingdon's disease, epilepsy, liver cirrhosis and stroke. In one embodiment, the condition is stroke.

Alzheimer's disease, SCI, oedema and stroke may be monitored by MRI so improvements in these conditions will lead to reduced areas of hyperintensity in the brain. Outcomes may also be followed by neurological assessment of motor and sensory function, with neurological improvements monitored, for example, by measurements in reflexes, muscle tone, sensation and improved mental state. An improvement, such as 25%-50%, for example 30%, as measured, for example, by MRI, would be significant for patients.

In a further embodiment there is provided the use of a compound or combination of compounds in the manufacture of a medicament for the treatment of spinal cord injury (SCI) or related injury to neurological tissue or for the treatment the secondary effects associated with SCI or related injury to neurological tissue, comprising selectively inhibiting the activity or expression of both matrix metalloproteinase MMP-9 (gelatinase-B) and metalloelastase MMP-12 after such SCI or related injury to neurological tissue.

In a further embodiment there is provided a method for the treatment of spinal cord injury (SCI) or related injury to neurological tissue or for the treatment of the secondary effects associated with SCI or related injury to neurological tissue, in a patient in need of such treatment, said method comprising administering to said patient a therapeutically effective amount of a compound or combination of compounds which selectively inhibiting the activity or expression of both matrix metalloproteinase MMP-9 (gelatinase-B) and metalloelastase MMP-12 after such SCI or related injury to neurological tissue. In one embodiment the related injury to neurological tissue is traumatic brain injury (TBI).

In a further embodiment there is provided a therapeutic kit of parts for use in treating spinal cord injury (SCI) or related injury to neurological tissue or for use in treating the secondary effects associated with SCI or related injury to neurological tissue, comprising
(i) a compound or combination of compounds which selectively inhibit the activity or expression of both matrix metalloproteinase MMP-9 (gelatinase-B) and metalloelastase MMP-12 after such SCI or related injury to neurological tissue and
(ii) instruction materials for teaching the administration of said compound or combination of compounds to a patient in need of such treatment.

The uses and methods described herein may also be used prophylactically, for example in association with brain or spinal surgery.

In another embodiment, there is provided a compound or combination of compounds which selectively inhibit the activity or expression of both matrix metalloproteinase MMP-9 (gelatinase-B) and metalloelastase MMP-12, for use in treating neuropathic pain.

In a further embodiment there is provided the use of a compound or combination of compounds in the manufacture of a medicament for the treatment of neuropathic pain, comprising selectively inhibiting the activity or expression of both matrix metalloproteinase MMP-9 (gelatinase-B) and metalloelastase MMP-12.

In a further embodiment there is provided a method for the treatment of neuropathic pain, in a patient in need of such treatment, said method comprising administering to said patient a therapeutically effective amount of a compound or combination of compounds which selectively inhibiting the activity or expression of both matrix metalloproteinase MMP-9 (gelatinase-B) and metalloelastase MMP-12.

In a further embodiment there is provided a therapeutic kit of parts for use in treating neuropathic pain, comprising
(i) a compound or combination of compounds which selectively inhibit the activity or expression of both matrix metalloproteinase MMP-9 (gelatinase-B) and metalloelastase MMP-12 after such SCI or related injury to neurological tissue and
(ii) instruction materials for teaching the administration of said compound or combination of compounds to a patient in need of such treatment.

In one embodiment, there is provided a method for the treatment of the secondary effects associated with SCI or related injury to neurological tissue, comprising selectively inhibiting the activity or expression of matrix metalloproteinases MMP-9 (gelatinase-B) and metalloelastase MMP-12 after such SCI or related injury to neurological tissue.

In one embodiment, there is provided a method for the treatment of the secondary effects associated with SCI or related injury to neurological tissue, said method comprising administering to a patient in need thereof a therapeutically effective amount of a compound or combination of compounds which selectively inhibiting the activity or expression of both matrix metalloproteinase MMP-9 (gelatinase-B) and metalloelastase MMP-12.

In one embodiment, the secondary effect associated with SCI or related injury to neurological tissue is SCI-induced oedema and/or neuropathic pain (NP).

Suitably, there is provided a method for treating neuropathic pain, said method comprising administering to a patient in need thereof a therapeutically effective amount of a compound or combination of compounds which selectively inhibiting the activity or expression of both matrix metalloproteinase MMP-9 (gelatinase-B) and metalloelastase MMP-12. Suitably, the compound or combination of compounds suppresses the proinflammatory pain markers IL-1β, TNF-α and/or II-6 after SCI or related injury to neurological tissue. Suitably, the compound or combination of compounds suppresses the proinflammatory pain markers IL-1β, TNF-α and/or II-6 after SCI or related injury to neurological tissue by at least 10%, 20%, 30%, 40%, 50%, 60% 70%, 80%, or 90%. Conveniently, the compound is AZD1236, or a pharmaceutically acceptable salt thereof. It is to be understood that suppression of the proinflammatory pain markers is relative to the levels in the subject prior to treatment with the compound or combination of compounds.

Suitably, there is a provided a method for treating SCI-induced oedema, said method comprising administering to a patient in need thereof a therapeutically effective amount of a compound or combination of compounds which selectively inhibiting the activity or expression of both matrix metalloproteinase MMP-9 (gelatinase-B) and metalloelastase MMP-12. Suitably, the compound or combinations of compounds reduces SCI-induced oedema by at least 10, 20, 30, 40, 50, 60, 70, 80, or 90%. Conveniently, the compound is AZD1236, or a pharmaceutically acceptable salt thereof. It is to be understood that the reduction is relative to the oedema in the subject prior to treatment with the compound or combination of compounds.

Other secondary effects associated with SCI or related injury to neurological tissue include reduction in BSCB breakdown and scarring at the SCI lesion site, preservation oflonger-term (neurological) function, wound treatment, the prevention of scarring (including combination treatment with decorin), and/or promotion of axon regeneration.

Suitably, there is provided a method for treating scarring in the CNS, said method comprising administering to a patient in need thereof a therapeutically effective amount of a compound or combination of compounds which selectively inhibiting the activity or expression of both matrix metalloproteinase MMP-9 (gelatinase-B) and metalloelastase MMP-12. Suitably, the compound or combinations of compounds reduces deposition of scar-related Sema-3A and CS-56 by at least 10, 20, 30, 40, 50, 60, 70, 80, or 90%. Conveniently, the compound is AZD1236, or a pharmaceutically acceptable salt thereof. It is to be understood that the reduction is relative to the scarring in the subject prior to treatment with the compound or combination of compounds.

Suitably, there is a provided a method for suppressing infiltration of blood-borne cells into the CNS, said method comprising administering to a patient in need thereof a therapeutically effective amount of a compound or combination of compounds which selectively inhibiting the activity or expression of both matrix metalloproteinase MMP-9 (gelatinase-B) and metalloelastase MMP-12. Suitably, the compound or combinations of compounds reduces blood-borne cells such as monocytes, neutrophils, granulocytes, macrophages and/or natural killer cells by at least 10, 20, 30, 40, 50, 60, 70, 80, or 90%. Conveniently, the compound is AZD1236, or a pharmaceutically acceptable salt thereof. It is to be understood that the reduction is relative to the infiltration of blood-borne cells in the subject prior to treatment with the compound or combination of compounds.

The uses and methods described herein are of particular use in treating human patients.

### Combination Therapies

In a particular embodiment, the compound or combination of compounds for use in treating spinal cord injury (SCI) or related injury to neurological tissue or for use in treating the secondary effects associated with SCI or related injury to neurological tissue, defined herein, may also be used in addition to conventional surgery and/or other chemotherapy. The other chemotherapy used in conjoint treatment may include one or more pharmaceutically-active agents used in the treatment of SCI or related injury to neurological tissue, for example, Lyrica (pregabalin) or methylprednisolone.

Such conjoint treatment may be achieved by way of the simultaneous, sequential or separate dosing of the individual components of the treatment. Such conjoint treatment employs the compound or compounds of this disclosure within the dosage range described herein and the other pharmaceutically-active agent within its approved dosage range.

Herein, where the term “conjoint treatment” is used it is to be understood that this refers to simultaneous, separate or sequential administration. In one aspect “conjoint treatment” refers to simultaneous administration. In another aspect “conjoint treatment” refers to separate administration. In a further aspect “conjoint treatment” refers to sequential administration. Where the administration is sequential or separate, the delay in administering the second component should not be such as to lose the beneficial effect of the combination.

According to a further aspect there is provided a pharmaceutical composition which comprises all the components of the conjoint treatment, in association with a pharmaceutically acceptable diluent or carrier.

In a further embodiment there is provided a compound or compounds for use as described herein wherein the use further comprises administration of an aquaporin-4 inhibitor (or inhibitors), such as TFP, PKAi or TGN-020.

In a further embodiment, there is provided a method for treating the secondary effects associated with SCI or related injury to neurological tissue, comprising selectively inhibiting the activity or expression of matrix metalloproteinases MMP-9 (gelatinase-B) and metalloelastase MMP-12 after such SCI or related injury to neurological tissue wherein the method further comprises administration of an aquaporin-4 inhibitor (or inhibitors), such as TFP, PKAi or TGN-020.

According to a further aspect there is provided a pharmaceutical composition which comprises all the components of the conjoint treatment, including an aquaporin-4 inhibitor (or inhibitors), in association with a pharmaceutically acceptable diluent or carrier.

### Routes of Administration

The compound or combination of compounds which selectively inhibit the activity or expression of both matrix metalloproteinase MMP-9 (gelatinase-B) and metalloelastase MMP-12, or a pharmaceutical composition/s comprising said compound or compounds, or any conjoint treatment, may be administered to a subject by any convenient route of administration, whether systemically, peripherally, topically or intrathecally (i.e. at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intraventricular, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In particular embodiments, oral or intrathecal delivery are provided, in particular intrathecal delivery. Such intrathecal delivery may use from 1/10^{th} to 1/50^{th} (for example, 1/20^{th} to 1/60^{th}) of the dose required for oral use, in particular 1/40^{th} of the oral dose to cause the same beneficial effects after SCI or related injury to neurological tissue. For example, if an oral dose of 200 mg/kg is used then intrathecal delivery may use a dose of 5 mg/kg. Suitably, the compound or combination of compounds are delivered intrathecally by an indwelling intrathecal catheter.

In a further embodiment the administration is provided for a period of up to one or two weeks, in particular for a short duration, for example for up to the first 3 days, or for one day, after SCI or related injury to neurological tissue.

In one embodiment, the administration is provided in one dose at the start of treatment. In another embodiment, the administration is provided continuously during treatment, for example by infusion.

### EXAMPLES

The following Examples are provided for illustration but do not limit the disclosure.

### Animals

All animal experiments were licensed by the UK Home Office and ethically approved by the University of Birmingham's Animal and Ethical Review Board. Experiments were carried out in strict accordance to the guidelines of the UK Animals Scientific Procedures Act, 1986 and the Revised European Directive 1010/63/EU and conformed to the guidelines and recommendation of the use of animals by the Federation of the European Laboratory Animal Science Associations and the Animal Research: Reporting of In Vivo Experiments (ARRIVE guidelines).

Wild-type adult male/female C57BL/6 mice (roughly equal proportions), 7-9-week-old and weighing between 20-30 g and male/female Sprague-Dawley rats (roughly equal proportions), 6-8-week-old and weighing between 170-220 g (purchased from Charles River, Margate, UK) were maintained under a 12-hour light/dark cycle in a pathogen-free facility with controlled temperature and humidity, and were fed ad libitum.

### Study Design

In general, all experiments were performed with n=6 animals/group and repeated on 2-3 independent occasions (total n=12-18 animals/group). No animals were excluded for any reason during this study. All animals were randomly allocated to the different experimental groups, ensuring equivalent representation of each animal cage in each experimental group. All treatments and experimental conditions were also masked to the investigators.

AZD1236 was delivered either orally or by intrathecal injection.

### Example 1

### Testing of AZD1236 in Dorsal Column (DC) Crush and Clip Compression (CC) Models of SCI in Mice

The DC crush model of SCI was selected due to being a moderate severity injury that transects the descending corticospinal tract and the ascending sensory gracile and cuneate tracts; the axons of which are derived from pyramidal motor neurons in layer V of the contralateral frontal motor neocortex and ipsilateral dorsal root ganglion neurons (DRGN), respectively. The injury response after DC lesion in mice has previously been characterised (Z. Ahmed, D. Bansal, K. Tizzard, S. Surey, M. Esmaeili, A. M. Gonzalez, M. Berry, A. Logan, Decorin blocks scarring and cystic cavitation in acute and induces scar dissolution in chronic spinal cord wounds. Neurobiol Dis 64, 163-176 (2014)), and others have demonstrated that it is a good model for analysis of regenerative outcomes. However, this model only shows post-injury sensitivity to pain for the first few hours after surgery and cannot be used to assess pain behaviours (S. Surey, M. Berry, A. Logan, R. Bicknell, Z. Ahmed, Differential cavitation, angiogenesis and wound-healing responses in injured mouse and rat spinal cords. Neuroscience 275, 62-80 (2014)). Therefore, we used a clip compression (CC) model of SCI, described below, to assess pain behaviours after treatment with AZD1236.

### In Vivo Experiments

For oral delivery of AZD1236, n=6 adult male C57BL6 mice/group (Charles River, Margate, UK) were randomly allocated to either: (1), Sham (control; partial laminectomy but no DC crush injury); (2), DC crush injury+vehicle (partial laminectomy followed by DC crush injury and injection of vehicle); (3), DC crush injury+100 mg/kg AZD1236; (4), DC crush injury+200 mg/kg AZD1236; and (5), DC crush injury+300 mg/kg AZD1236. For intrathecal (it) delivery of AZD1236, adult male C57BL6 mice (25-30 g) (Charles River, Margate, UK) were randomly allocated to either: (1), Sham (control; partial laminectomy but no DC crush injury); (2), DC crush injury+vehicle (partial laminectomy followed by DC crush injury and injection of vehicle); (3), DC crush injury+2.5 mg/kg AZD1236; (4), DC crush injury+5 mg/kg AZD1236; and (5), DC crush injury+10 mg/kg AZD1236. For intrathecal delivery an indwelling atlanto-occipital intrathecal catheter was inserted into the intrathecal space, as described previously (F. A. Oladosu, B. P. Ciszek, S. C. O'Buckley, A. G. Nackley, Novel intrathecal and subcutaneous catheter delivery systems in the mouse. J Neurosci Methods 264, 119-128 (2016)). Briefly, with mice in the sternal recumbency a small incision was made at the nape of the neck followed by detachment of the muscles on either side of the external occipital crest to expose the A-O membrane. The membrane was incised and a 6 cm Alzet mouse intrathecal catheter (Alzet, Cupertino, Calif., USA) and polyurethane segments were gently guided 2.5 cm into the intrathecal space. The catheter was secured using 4-0 silk sutures (Oasis Medical, IL) and the exposed end of the catheter was sealed off with a stainless-steel plug and affixed to the upper back. Animals were injected immediately with vehicle or AZD1236 followed by a 10 μl PBS catheter flush. Injections were repeated every twice daily for the first 3 days after injury and drugs and vehicle reagents were delivered over 1 min time period using a Hamilton microlitre syringe (Hamilton Co, USA). Where specified intact animals with no surgical procedures were also used. All lesions were administered under 5% isofluorane induced inhalation anaesthesia, with 1.51/min O₂. Pre- and post-injury analgesia was also provided.

DC crush injury was administered bilaterally at the T8 vertebral level as described previously (S. Surey, M. Berry, A. Logan, R. Bicknell, Z. Ahmed, Differential cavitation, angiogenesis and wound-healing responses in injured mouse and rat spinal cords. Neuroscience 275, 62-80 (2014); M. L. Read, S. Mir, R. Spice, R. J. Seabright, E. L. Suggate, Z. Ahmed, M. Berry, A. Logan, Profiling RNA interference (RNAi)-mediated toxicity in neural cultures for effective short interfering RNA design. J Gene Med 11, 523-534 (2009)). Briefly, calibrated watchmaker's forceps were separated by 0.5 mm and inserted through the dorsal cord meninges to a depth of 0.45 mm in mice and separated by 1 mm and inserted through the dorsal cord meninges to a depth of 1.0 mm in rats, and the DC were crushed for 3 seconds. AZD1236 or vehicle was administered twice daily by oral gavage or intrathecally, either immediately for the first 3 days or 24 hours after injury and treating twice daily until day 4 (i.e. 3 days of AZD1236 dosing). All experiments were performed with n=6 mice/group and repeated on at least 2-3 independent occasions (total=12-18 animals/group/test).

CC SCI was administered at the T7-T8 vertebral level after exposure of T6-T9 by laminectomy. The aneurysm clip applicator was oriented in a bilateral direction and an aneurysm clip with a closing force of 24 g was applied extradurally for 60 s, as described previously (A. S. Rivlin, C. H. Tator, Effect of duration of acute spinal cord compression in a new acute cord injury model in the rat. Surg Neurol 10, 38-43 (1978):E. Esposito, I. Paterniti, E. Mazzon, T. Genovese, M. Galuppo, R. Meli, P. Bramanti, S. Cuzzocrea, MK801 attenuates secondary injury in a mouse experimental compression model of spinal cord trauma. BMC Neurosci 12, 31 (2011)).

The urinary bladders were manually emptied twice daily until bladder function was regained. Adult male C57BL6 mice (25-30 g) (Charles River, Margate, UK) were randomly allocated to either: (1), Sham (control; laminectomy but no CC SCI); (2), CC SCI+vehicle; (3), CC SCI+200 mg/kg AZD1236 (oral); (4), CC SCI+5 mg/kg AZD1236 (it). All experiments were performed with n=6 mice/group and repeated on 3 independent occasions (total=18 mice/group/test).

The efficacy of oral AZD3342 in the DC injury model in adult Sprague-Dawley rat was also assessed. Animals were divided into 6 groups: (1), Sham (control); (2), DC crush injury+Vehicle; (3), DC crush injury+15 mg/kg AZD3342; (4), DC crush injury+75 mg/kg AZD1236; (5), DC crush injury+375 mg/kg AZD3342; and (6), DC crush injury+Melatonin. Animals were dosed twice daily with oral AZD3342 or vehicle with the experimenters masked to the treatment conditions. These experiments were performed with n=6 rats/group and repeated on 2-3 independent occasions (total n=12-18 rats/group/test). All tool and clinically relevant MMP inhibitors were delivered and preoptimized in our injury models, as indicated in the table below:

TABLE 1
MMP inhibitor concentrations used in this study.

| | | | Doses tested | Route and |
|---|---|---|---|---|
| Inhibitor | Source | Activity | mg/kg | Frequency |
| AZD1236 | AstraZeneca, | MMP-9 and -12 | 100, 200 | Oral, twice daily |
| | Cambridge, UK | | and 300 | It, twice daily |
| | | | 2.5, 5 | |
| | | | and 10 | |
| AZD3342 | AstraZeneca, | MMP-9 and -12 | 15, 75 | Oral, twice daily |
| | Cambridge, UK | | and 375 | |
| GM6001 | Tocris, Oxford, | MMP-1, -2, -3, -8 | 100-500 | IP, twice daily ¹ |
| | UK | and -9 | | |
| SB-3CT | Tocris, Oxford, | MMP-2 and -9 | 10, 20, 25, | IP at 2 and 4 hours |
| | UK | | 30, 35 | after injury then |
| | | | | once daily ² |
| MMP-9 | Merck, Watford, | MMP-9 | 100-500 | IP, twice daily |
| Inhibitor I | UK | | | |
| MMP408 | Merck, Watford, | MMP-12 | 100, 200 | IP, twice daily |
| | UK | | and 300 | |
| ND-378 | MedKoo, | MMP-2 | 15, 25, 35, | IP at 24 hours, SC 1 |
| | Mirrisville, | | 50, 60 | hour after first IP |
| | NC, USA | | | dose, then SC doses |
| | | | | once daily ³ |
| Melatonin | Tocris, Oxford, | Aquaporin-4 | 50-400 | IP, twice daily ⁴ |
| | UK | | | |
| Minocycline | Tocris, Oxford, | No effect on | 50 | 50 mg for 2 days then |
| | UK | MMP activity | | 25 mg/kg ⁵ |
| Riluzole | Tocris, Oxford, | GABA uptake | 8 | IP, 12 hours pre-SCI |
| | UK | inhibitor | | and twice daily for |
| | | | | 3 days ⁶ |
| Glibenclamide | Tocris, Oxford, | Sur1-regulated | 0.01 | IP, loading dose |
| | UK | NCCa-ATP | | of 0.01 mg/kg then |
| | | | | 200 ng/hour for |
| | | | | 7 days ⁷. |

¹ Noble, L. J., Donovan, F., Igarashi, T., Goussev, S. & Werb, Z. J Neurosci 22, 7526-7535 (2002).
² Cui, J., et al. Mol Neurodegener 7, 21 (2012).
³ Gao, M., et al. ACS Chem Neurosci 7, 1482-1487 (2016).
⁴ Li, C., et al. Neural Regen Res 9, 2205-2210 (2014).
⁵ Wells, J. E., Hurlbert, R. J., Fehlings, M. G. & Yong, V. W. Brain 126, 1628-1637 (2003).
⁶ Caglar, Y. S., et al. World Neurosurg 114, e247-e253 (2018).
⁷ Simard, J. M., et al. Exp Neurol 233, 566-574 (2012).
Unless referenced, all doses have been tested in this study. IP = intraperitoneal, It = intrathecal, SC = subcutaneous. Sur1-regulated NCCa-ATP = sulfonylurea receptor 1-regulated NC_{Ca}-ATP channels.

### Cholera Toxin B Labeling of Axons

For retrograde tracing of axons, 1% cholera toxin B (CTB) (#104, List Biologicals Laboratories, Campbell, Calif., USA) was injected into the sciatic nerve after exposure at mid-thigh level, using glass microneedles. The skin was re-sutured and the animals allowed to recover for 1 week prior to killing animals using rising concentrations of CO₂ followed by intracardiac perfusion with 4% paraformaldehyde (TAAB laboratories, Berkshire, UK). CTB labelled axons were detected by immunohistochemistry as described later.

### Quantification of SCI-Induced Oedema

The lesion site+3 mm either side for mice and the lesion site+5 mm either side for the rat DC model were dissected out and the water content of the spinal cord (as a measure of oedema) was determined at 3 days after DC and CC SCI, as described previously (S. Li, C. H. Tator, Effects of MK801 on evoked potentials, spinal cord blood flow and cord edema in acute spinal cord injury in rats. Spinal Cord 37, 820-832 (1999)). Spinal cords containing the lesion site were weighed on aluminium foil, dried at 105° C. for 24 hr, and re-weighed. The percent water content was calculated as: water content (%)=[(wet weight-dry weight)/wet weight]×100%.

Quantitative RT-PCR (qRT-PCR)

The lesion site+5 mm either side (DC and CC SCI, n=6 mice/rats/group, 2 independent repeats (total n=12 mice/rats/group)) was dissected and rapidly frozen in liquid N₂ and stored at −80° C. until required. Total RNA was extracted from spinal cords at appropriate time-points after injury with or without treatment using TRIzol reagent according to the manufacturer's instructions (Invitrogen). The levels of mRNA of MMP-9, MMP-12, IL-1β, TNF-α and IL-6 were determined using pre-validated mouse primer sequences from complimentary DNA prepared from extracted mRNA and qRT-PCR was performed using a LightCycler PCR machine (Roche, Burgess Hill, UK) (M. L. Read, S. Mir, R. Spice, R. J. Seabright, E. L. Suggate, Z. Ahmed, M. Berry, A. Logan, Profiling RNA interference (RNAi)-mediated toxicity in neural cultures for effective short interfering RNA design. J Gene Med 11, 523-534 (2009)). Primer sequences for mouse included: MMP-9− cat no. 4331182, Mm0044299_ml; MMP-12− cat no. 4331182, Mm00500554_ml; IL-1β− cat no. 4331182, Mm00434228_ml; TNF-α− cat no. 4331182, Mm00443258_ml; and IL-6− cat no. 4331182, Mm00446190_ml; for rat included: MMP-9− cat no. 4331182, Rn00579162_ml; MMP-12− cat no. 4331182, Rn00588640_ml; IL-1β3− cat no. 4331182, Rn00580432_ml; TNF-α− cat no. 4331182, Rn01525859_g1; and IL-6− cat no. 4881182, Rn01410330_ml (all from ThermoFisher Scientific, Leicestershire, UK). Fold changes were computed using the ΔΔCt method (M. L. Read, S. Mir, R. Spice, R. J. Seabright, E. L. Suggate, Z. Ahmed, M. Berry, A. Logan, Profiling RNA interference (RNAi)-mediated toxicity in neural cultures for effective short interfering RNA design. J Gene Med 11, 523-534 (2009).

### Detection of MMP-9 and MMP-12 Levels in the Spinal Cord

The lesion site+3 mm either side for mouse (DC and CC SCI) was dissected (n=6 mice/rats/group, 2 independent repeats, total n=12 mice/rats/group) and rapidly frozen in liquid N₂ and stored at −80° C. until required. Samples were then homogenized in ice-cold lysis buffer containing protease inhibitors (all from Sigma, Poole, UK). MMP-9 (ab253227, Abcam, Cambridge, UK) and MMP-12 (ab213878, Abcam) was detected using ELISA kits obtained from Abcam, following the manufacturer's instructions.

### Detection of MMP-9 and MMP-12 Enzyme Activity

The lesion site+3 mm either side for mouse (DC and CC SCI) and lesion site+5 mm either side for rat was dissected (n=6 mice/rats/group, 2 independent repeats, total n=12 mice/rats/group) and rapidly frozen in liquid N₂ and stored at −80° C. until required. The enzymatic activity of MMP-9 and MMP-12 was determined in 96-well plates using the SensoLyte 520 MMP-9 and MMP-12 fluorimetric assay kit, according to the manufacturer's instructions (AnaSpec, Fremont, Calif., USA). Fluorescence intensity was measured using a Synergy H1 microplate reader (BioTek UK, Swindon, UK) at Ex/Em=490/520 nm.

### In Situ Zymography Followed by Localization of Astrocytes

Animals were killed by CO₂ overdose intracardially perfused with warm PBS and unfixed tissues were harvested. The lesion site+3 mm either side for mice and lesion site+5 mm either side was dissected (n=6 mice/rats/group, 2 independent repeats, total n=12 mice/rats/group) and immediately blocked up in optimal cutting temperature compound (OCT; Miles Inc, Elkhart, Ill., USA) and stored at −80° C. until required for cryostat sectioning. In situ zymography was performed as described previously (Z. Ahmed, R. G. Dent, W. E. Leadbeater, C. Smith, M. Berry, A. Logan, Matrix metalloproteases: degradation of the inhibitory environment of the transected optic nerve and the scar by regenerating axons. Mol Cell Neurosci 28, 64-78 (2005)).

Briefly, 15 μm-thick unfixed frozen longitudinal sections of the spinal cord were cut on a cryostat and incubated at 25° C., for 24 h in 50 mM Tris, pH 7.4, 150 mM NaCl, 5 mM CaCl2, 0.2 mM sodium azide and 40 ug/ml fluorescein conjugated DQ™ gelatin (Molecular Probes). Upon proteolysis, the highly fluoresceinated gelatin residues became separated and fluorescence resulted. Control sections were incubated either without DQ™ gelatin or incubated with 50 μM 1,10-phenanthroline (Sigma, Poole, UK) to block MMP activation. Sections were then fixed in 4% paraformaldehyde (TAAB Laboratories), washed in PBS and incubated with anti-glial fibrillary acidic protein (GFAP) antibodies (SAB5700611, 1:400 dilution, GFAP; Sigma) for 1 hour at room temperature in a humidified chamber. Sections were then washed in PBS and incubated with appropriate secondary antibodies conjugated with Texas Red (Invitrogen). After final washes in PBS, sections were mounted in Vectashield with DAPI (Vector Laboratories, Loughborough, UK) and viewed with a Zeiss Axioplan 2 fluorescence microscope, equipped with an AxioCam HRc and running Axiovision software (Zeiss, Hertfordshire, UK).

### Immunohistochemistry

After killing animals by exposure to rising concentrations of CO₂, mice were intracardially perfused with 4% formaldehyde (TAAB Laboratories, Berkshire, UK) in 0.1M phosphate buffered saline (PBS). The lesion site+5 mm either side were dissected out, post-fixed in 4% formaldehyde and subjected to a graded series of sucrose solutions for cryoprotection. Spinal cords were blocked in OCT mounting compound (TAAB laboratories), sectioned longitudinally at 15 μm-thick using a cryostat (Brights Instruments, Huntingdon, UK) before being collected on charged glass slides (ThermoFisher Scientific, Loughborough, UK) and kept at −20° C. until required. Slides were numbered consecutively and sections from the middle of the lesion site were chosen for all immunohistochemical analyses, as described by previously (Surey S, Berry M, Logan A, Bicknell R, Ahmed Z. Differential cavitation, angiogenesis and wound-healing responses in injured mouse and rat spinal cords. Neuroscience 2014; 275: 62-80).

The integrity of the BSCB was assessed using albumin immunoreactivity in the spinal cord at 3 days after injury, as a surrogate marker of BSCB disruption. Sections were thawed at room temperature and washed in PBS before blocking endogenous peroxide in H₂O₂. Sections were then permeabilization in 0.1% Triton X-100 in PBS for 10 min at RT, blocked in 4% serum in PBS and incubated overnight with rabbit anti-albumin primary antibodies (ab271979; 1:1000 dilution, Abcam). Sections were then washed in PBS and incubated for 1 hour at room temperature with HRP-labelled anti-rabbit secondary antibody, followed by washes in PBS and incubation with avidin-biotin conjugate for 30 mins. Colour was developed using the 3,3′-Diaminobenzidine (DAB) substrate kit, dehydrated through a graded series of alcohols, cleared in Histoclear and coverslips mounted in Vectamount (all from Vector Labs).

Fluorescent immunohistochemistry was performed as described previously (S. Surey, M. Berry, A. Logan, R. Bicknell, Z. Ahmed, Differential cavitation, angiogenesis and wound-healing responses in injured mouse and rat spinal cords. Neuroscience 275, 62-80 (2014)). Briefly, longitudinal spinal cord sections were washed in PBS, followed by incubation in PBS containing 1% (v/v) Triton X-100 (Sigma) to permeabilise cells. Sections were then blocked for 30 min at room temperature (RT) using PBS containing 0.05% (w/v) bovine serum albumin (Sigma, Poole, UK) and 0.05% Tween-20 (Sigma) and incubated overnight at 4° C. in a humidified chamber with appropriate antibodies. Laminin was detected at 4 weeks after DC injury and treatment using a rabbit polyclonal anti-laminin primary antibody (ab11575; 1:400 dilution, Abcam). Macrophages were detected with a rabbit polyclonal anti-CD68 antibody (ab1525212; 1:500 dilution, Abcam); microglia were detected using a rabbit polyclonal antibody to CD11b (ab128797, 1:500 dilution, Abcam); GFAP was detected using a polyclonal anti-GFAP antibody (SAB5700611, 1:400 dilution, Sigma) were all detected at 10 days after DC injury and treatment. Semaphorin-3A (Sema-3A) and chondroitin sulphate proteoglycan (CSPG) was detected using monoclonal anti-CS-56 antibody (C8035; 1:200 dilution, Sigma) at 7 days after injury. This timepoint was chosen since a number of studies have indicated that CSPGs are observed around the lesion site at 7-d days after injury (for example Tang X, Davies J E, Davies S J. Changes in distribution, cell associations, and protein expression levels of NG2, neurocan, phosphacan, brevican, versican V2, and tenascin-C during acute to chronic maturation of spinal cord scar tissue. J Neurosci Res 2003; 71(3): 427-44). CTB labelled axons in the mouse were detected using a goat polyclonal anti-CTB antibody (#703; 1:1000 dilution, List Biological Labs) at 6 weeks after DC injury.

Sections were then washed in PBS before incubation for 1 h at room temperature with Alexa488 and Alex595-conjugated secondary antibody (all used at 1:400 dilution; Invitrogen). Sections were then washed in PBS and coverslips mounted using Vectashield mounting medium (containing DAPI) (Vector Laboratories, Peterborough, UK)).

Negative controls were included in each run where primary antibodies were omitted and these slides were used to set the background threshold levels prior to image capture. Sections (fluorescent and DAB stained) were viewed using and Axioplan 2 fluorescent microscope equipped with an AxioCam HRc and Axiovision software (all from Zeiss, Hertfordshire, UK).

### Quantification of Immunofluorescence

All analyses were performed by investigators masked to the experimental groups. Relative fluorescent staining intensity was calculated by image analysis as described previously (Surey S, Berry M, Logan A, Bicknell R, Ahmed Z. Differential cavitation, angiogenesis and wound-healing responses in injured mouse and rat spinal cords. Neuroscience 2014; 275: 62-80). Briefly, photomicrographs taken at 5× magnification using the same standardized exposure settings throughout for each antibody were thresholded and the mean integrated intensity of pixels/unit area for each antibody was recorded Using ImageJ (NIH, USA) from n=12 mice/antibody.

Regeneration of DC axons were quantified from sagittal spinal cord sections form the whole series of tissue for each mouse (total n=12 mice/group). CTB intensity was quantified using ImageJ software at set distances rostral to the injury center and expressed as a percentage of the CTB intensity caudal to the injury site to control for variations in tracing efficiency.

### Primary Microglia Cultures and Treatments

Primary mouse microglia from 6-8-week-old C57BL/6 mouse brains were prepared according to a previously described protocol (Moussaud S, Draheim H J. A new method to isolate microglia from adult mice and culture them for an extended period of time. J Neurosci Methods 2010; 187(2): 243-53). Briefly, the meninges of brains were dissected out, finely minced enzymatically digested using 20 units/ml Papain (all from Sigma). After incubation for 90 min at 37° C., the suspension was centrifuged at 200 g for 7 min and the pellet resuspended in 0.5 mg/ml DNase I (Roche, Manheim, Germany) and triturated with fire-polished Pasteur pipettes of decreasing diameters. The homogenate was then filtered through a 70 μm cell strainer (Beckton Dickinson, Watford, UK), and centrifuged through a Percoll (GE Healthcare, Amersham, UK) gradient. Finally, the cell suspension was resuspended in DMEM/F12 medium supplemented with 10% FBS (all from Invitrogen) and 5 ng/ml of granulocyte colony and macrophage stimulating factor (GM-CSF) (#415-ML, R&D Systems. Watford, UK), plated out in T75 cell culture flasks (Beckton Dickinson) precoated with poly-L-lysine, and maintained at 37° C. and 5% CO₂ for approximately 2 weeks. When the cells become confluent, microglia detach and migrate to the medium-air interface, floating and proliferating. The supernatant of the flask was then collected without prior shaking to remove the microglia growing on a mixed glial culture base and centrifuged at 200 g for 7 min. The purity of microglia was determined from each flask by immunocytochemistry for CD11b and confirmed as 95% pure. Before each experiment, microglia were cultured in DMEM/F12 without GM-CSF for at least 3 d.

Microglia were used for all experiments with an in vitro age between 15-20 d in vitro (DIV) and were seeded on glass coverslips in 24-well plates at a density of 3×10⁴ cells/well. Microglia were exposed to serum-free media for 24 h before exposing the cells to different concentrations of lipopolyscaccharide (LPS) in preliminary experiments to determine optimal concentrations required to maximally activate our primary microglial cultures. This was determined as 10 ng/ml. For the microglial activation assay, cells were exposed to serum-free medium for 24 h before being exposed to LPS, with or without different concentrations of AZD1236, GM6001, SD2590 and MMP-9 Inhibitor I (all used at 10-500 ng/ml) for a further 24 h. Cell culture supernatant was then collected, centrifuged to remove cell debris and subjected to ELISA to determine the concentrations of TNF-α, IL-1β and IL-6. Experiments were performed in triplicate wells and repeated on 3 independent occasions (n=9 wells/treatment).

### ELISA to Determine Cytokine Levels

TNF-α (#MTA00B), IL-1β (#MLBOOC) and IL-6 (#M6000B) were determined using commercially available kits from R&D Systems, following the manufacturer's instructions.

### Cell Culture

J774A.1 cells (#TIB-67; ATCC, Middlesex, UK) were cultured in DMEM supplemented with 10% FBS, 100 U/ml penicillin, 100 μg/ml streptomycin, and 0.25 μg amphotericin B. Cells were seeded in T75 tissue-culture flasks and maintained at 37° C. and 5% CO₂. RAW 264.7 cells (#TIB-71; ATCC) were maintained in DMEM containing 10% FBS.

### Peritoneal Macrophage Preparation

Resident peritoneal cells were harvested from adult C57BL/6 mice (6-8 weeks old) by injection of 10 ml of PBS using a 21G needle into the peritoneal cavity and harvesting the cell suspension, as described previously (Rosas M, Davies L C, Giles P J, Liao C T, Kharfan B, Stone T C, et aL. The transcription factor Gata6 links tissue macrophage phenotype and proliferative renewal. Science 2014; 344(6184): 645-8). Peritoneal cells were then centrifuged at 100×g for 10 min and the cell pellet resuspended in DMEM/F12 medium. Cell were incubated at 37° C., for 2 h and non-adherent cells were removed by gentle washing. Cells were expanded in DMEM/F12 medium containing 10% FBS. For migration assays, cells were grown in RPMI containing 0.02% BSA for 24 h to obtain quiescent cells prior to using them in Transwell migration assays as described below.

### Transwell Migration Assays

Migration assays were performed with J774A.1, RAW 264.7 and primary mouse peritoneal macrophages in 6.5 mm Transwell plates (ThermoFisher) with 8 μm pore inserts, as described previously (Green et al., 2012). Briefly, inserts were coated with rat tail type I collagen and 1×10⁵ cells (J774A.1, RAW 264.7 or primary mouse macrophages) were resuspended in chemotaxis buffer (RMPI 1640 plus 0.02% BSA, (termed RPMI from herein)) and added to the upper chamber and incubated with migration medium, with or without known chemotactic factors MCP-1 (100 ng/ml) and PMA (100 nM), or AZD1236, GM6001, SD2590 and MMP-9 Inhibitor I (all used at 1, 10, 100, 1000 ng/ml) added to the lower chamber. Cells were allowed to migrate through the insert membrane for 3 h at 37° C., before washing inserts with PBS. Non-migrating cells remaining on the upper surface were removed with a cotton swab whilst the migrated cells on the insert were fixed, stained with Diff-Quick (#26096, Electron Microscopy Science, Hatfield, UK), and mounted on glass slides. Migration was measured visually by counting using a light microscope at 40× magnification. The mean number of cells in 10 random fields were calculated for each treatment by an experimenter masked to the treatment conditions. A migration index was calculated by dividing the number of cells that migrated in response to the chemokine by the number of cells that migrated randomly (RPMI medium) with a reference index>1, indicating chemotaxis.

### Electrophysiology

Compound action potentials (CAP) were recorded at 6 w weeks after DC injury and treatment, as described previously—for example B. C. Hains, C. Y. Saab, A. C. Lo, S. G. Waxman, Sodium channel blockade with phenytoin protects spinal cord axons, enhances axonal conduction, and improves functional motor recovery after contusion SCI. Exp Neurol 188, 365-377 (2004).

Briefly, the experimenter was masked to the treatment status of the animals and the CAP amplitude was calculated between the negative deflection after the stimulus artifact and the next peak of the wave. CAP area was also calculated by rectifying the CAP component (full-wave rectification) and measuring its area. To confirm our recordings and that a CAP could not be recorded, the dorsal half of the spinal cord was transected after each experiment between the stimulating and recording electrodes.

### Functional Tests

Functional testing after DC lesion and treatment was carried out as described previously (N. D. Fagoe, C. L. Attwell, R. Eggers, L. Tuinenbreijer, D. Kouwenhoven, J. Verhaagen, M. R. Mason, Evaluation of Five Tests for Sensitivity to Functional Deficits following Cervical or Thoracic Dorsal Column Transection in the Rat. PLoS One 11, e0150141 (2016)).

Briefly, animals (n=18/group) were first trained to master traversing a horizontal ladder for 1 w before functional testing. Baseline parameters were established by performing tests at 2-3d before injury. Animals were then tested at 2d, 1 w, 2 w, 3 w, 4 w, 5 w and 6 w after DC injury+treatment. Experiments were performed by an observer masked to the treatment conditions in the same order and time of day with each test performed for 3 individual trials.

Horizontal ladder test: This tests the animals locomotor function and is performed on a 0.9-meter-long horizontal ladder with a diameter of 15.5 cm and randomly adjusted rungs with variable gaps of 3.5-5.0 cm. Animals were assessed traversing the ladder and the left and right rear paw slips were recorded along with the total number of steps and the mean error rate as: the number of slips/total number of steps.

Tape removal test (sensory function): The tape removal test determines touch perception from the left hind paw. After holding animals with both hind-paws extended, the time it took for the animal to detect and remove a piece of tape of 15×15 mm (Kip Hochkrepp, Bocholt, Germany) affixed to the palm of the left hind-paw was recorded and used to calculate the mean sensing time.

### Assessment of Neuropathic Pain (NP)

Mechanical allodynia was measured in mice using a series of von Frey filaments (0.25 g-15 g) applied to the plantar surface of the hind-paw by an experimenter masked to the treatment conditions and withdrawal of the paw was recorded as a positive response (F. Nasirinezhad, S. Gajavelli, B. Priddy, S. Jergova, J. Zadina, J. Sagen, Viral vectors encoding endomorphins and serine histogranin attenuate neuropathic pain symptoms after spinal cord injury in rats. Mol Pain 11, 2 (2015)).

The hair with the lowest force required to elicit a paw withdrawal response was recorded. Both hind paws were tested with a 5 min rest between testing of the opposite paw.

Thermal hyperalgesia was tested using established methods of plantar heat sensitivity tests described previously (S. Surey, M. Berry, A. Logan, R. Bicknell, Z. Ahmed, Differential cavitation, angiogenesis and wound-healing responses in injured mouse and rat spinal cords. Neuroscience 275, 62-80 (2014)). Briefly, animals were acclimatized in clear Perspex compartments before surgery post injury and treatment data were collected at the same time of day to ensure consistency. Animals were also acclimatized to the clear Perspex compartments for 5 min before testing commenced. An infra-red heat source (Harvard Apparatus, Kent, UK) was applied to the plantar surface of the hindlimbs once the animals were stationary. The reaction time of paw withdrawal was recorded for five separate tests for each hind limb with a 30-s interval before re-testing on the same paw. The middle three scores for the hind limbs were averaged to produce a single score for each animal.

Cold allodynia was measured by an experimenter masked to the treatment conditions as the number of foot withdrawal responses after application of an acetone drop to the plantar surface of the paw (F. Nasirinezhad, S. Gajavelli, B. Priddy, S. Jergova, J. Zadina, J. Sagen, Viral vectors encoding endomorphins and serine histogranin attenuate neuropathic pain symptoms after spinal cord injury in rats. Mol Pain 11, 2 (2015)). Testing was repeated five times with an interval of 5 min between each test and the response frequency to acetone was expressed as a percent response frequency ([number of paw withdrawals/number of trials]×100).

### Statistical Analysis

Statistical significance was calculated from sample means by one-way analysis of variance (ANOVA) with post-hoc Dunnett's method using SPSS Statistics 19 (IBM, New York, USA). For the horizontal ladder crossing and tape removal tests, data was analysed as described previously (Tuxworth RIT, M. J.; Anduaga, A-M.; Hussien-Ali, A.; Chatzimatthaiou, S.; Longland, J.; Thompson, A. M.; Almutiri, S.; Alifragis, P.; Kyriacou, C. P.; Kysela, B.; Ahmed, Z. Attenuating the DNA damage response to double-strand breaks restores function in models of CNS neurodegeneration. Brain Communications 2019; 1(1): fcz005) using R package (www.r-project.org).

Briefly, for the ladder crossing test, whole time-course of lesioned and sham-treated animals were compared using binomial generalized linear mixed models (GLMM). Binomial GLMMs were fitted in R using package/me4 with the g/merfunction. P values were then calculated using parametric bootstrap. For the tape removal test, linear mixed models (LMM) were calculated by model comparison in R using the package pbkrtest, with the Kenward-Roger method (N. D. Fagoe, C. L. Attwell, R. Eggers, L. Tuinenbreijer, D. Kouwenhoven, J. Verhaagen, M. R. Mason, Evaluation of Five Tests for Sensitivity to Functional Deficits following Cervical or Thoracic Dorsal Column Transection in the Rat. PLoS One 11, e0150141 (2016).

For pain behaviour tests, data were compared among groups using two-way ANOVA with repeated measures followed by Bonferroni post hoc test.

All results are presented as mean±standard error of the mean (SEM) and error bars in the figures represent SEM.

### Results

MMP-9 and MMP-12 are Acutely Upregulated after DC Injury

The spatio-temporal expression pattern of MMP-9 and MMP-12 in a mouse model of SCI were studied where the dorsal columns (DC) are bilaterally injured at the level of thoracic (T)8 (Surey S, Berry M, Logan A, Bicknell R, Ahmed Z. Differential cavitation, angiogenesis and wound-healing responses in injured mouse and rat spinal cords. Neuroscience 2014; 275: 62-80).

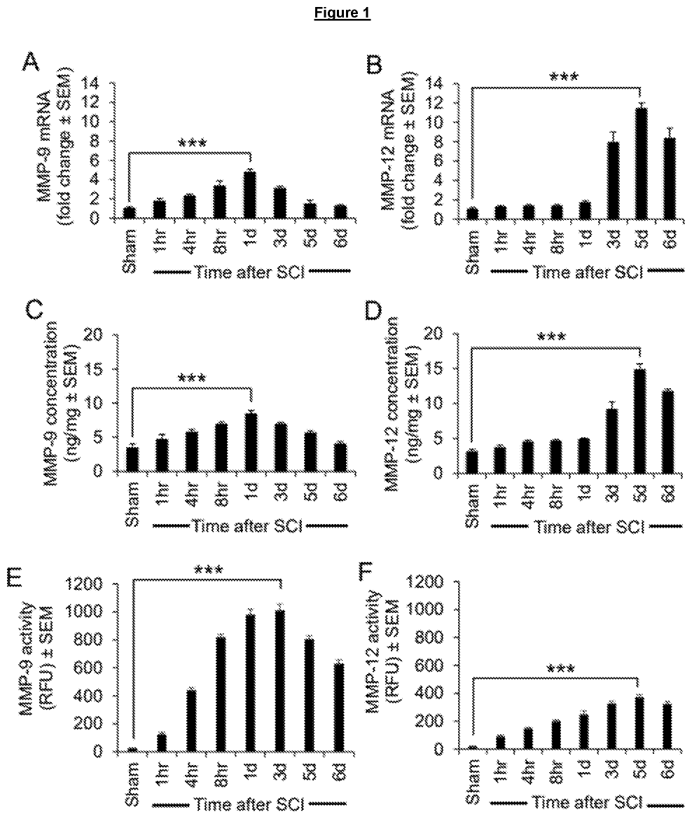

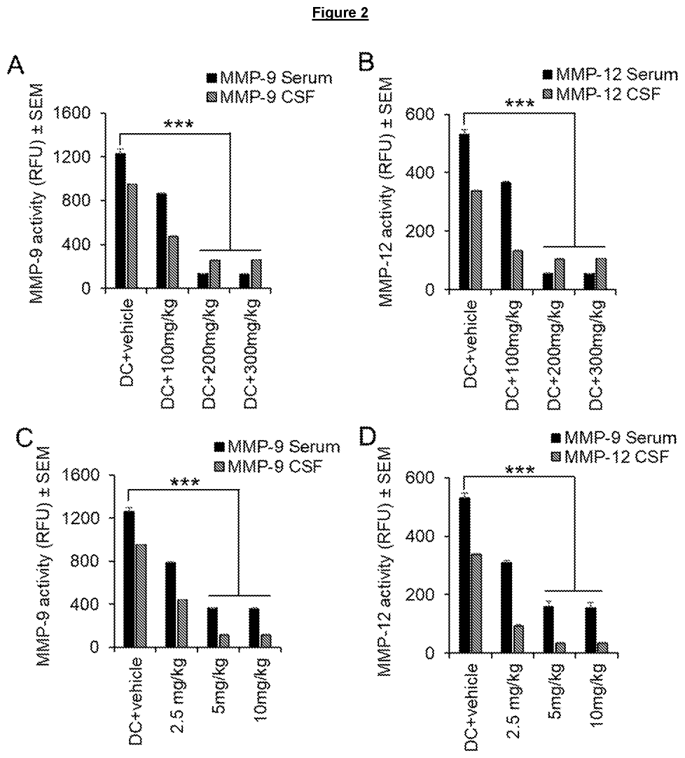

To confirm the time-course of the post injury rise in MMP-9 and MMP-12, qRT-PCR was used. In line with levels of MMP-9 in human SCI (Casha S, Rice T, Stirling D P, Silva C, Gnanapavan S, Giovannoni G, et aL. Cerebrospinal Fluid Biomarkers in Human Spinal Cord Injury from a Phase II Minocycline Trial. J Neurotrauma 2018; 35(16): 1918-28), MMP-9 mRNA in mice was also upregulated within 1 hr after injury by 1.9-fold and peaked at 24 hours rising to 4.8-fold, compared to sham-treated mice (FIG. 1A). The levels of MMP-9 declined thereafter and returned to control sham-treated levels by 6d after DC injury (FIG. 1A). Levels of MMP-12 however, did not rise until 3d after DC injury, peaking at 5d where MMP-12 levels were 11.5-fold higher compared to sham-treated mice (FIG. 1B). MMP-12 levels declined at 6d after DC injury (FIG. 1B). Protein levels of MMP-9 (FIG. 1C) and MMP-12 (FIG. 1D) mirrored their mRNA levels, whilst the relative activity of these enzymes (FIGS. 1E and F) correlated with changes in their expression levels over time. These results demonstrate that both MMP-9 and MMP-12 mRNA protein levels and enzyme activity peak within the first 5d after injury and thereafter decline.

### AZD1236 Effectively Suppresses MMP-9 and MMP-12 Activity in the Spinal Cord, Serum and CSF

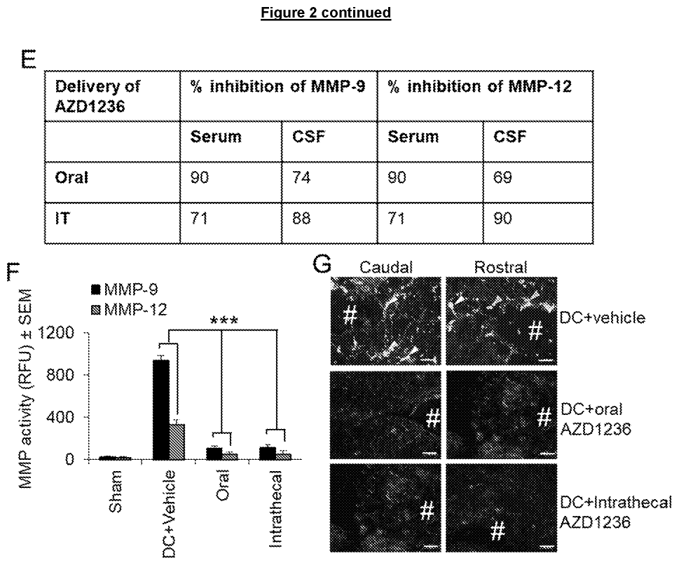

The levels of MMP-9 and MMP-12 activity in serum and CSF after DC injury and dosing with AZD1236 was determined. MMP-9 and MMP-12 activity were suppressed by 90±2% and 90±3% in serum (FIGS. 2A and E) and by 74±2% and 69±3% in CSF (FIGS. 2B and E), respectively, after oral delivery of AZD1236 (200 mg/kg dose). Following intrathecal delivery, MMP-9 and MMP-12 activity were respectively suppressed by 71±2% and 71±1% in serum (FIGS. 2C and E) and by 88±2% and 90±1% in CSF (FIGS. 2D and E). These results demonstrate that the lowest most effective dose of AZD1236 was 200 mg/kg by oral administration and 5 mg/kg by intrathecal injection. In the spinal cord, MMP-9 and MMP-12 activity were barely detectable in sham-treated cords (FIG. 2F), however, DC injury caused a 98±2% and 95±2% respective increases in MMP-9 and MMP-12 activity (FIG. 2F). The lowest most effective dose of AZD1236 suppressed MMP-9 activity in spinal cord tissues by 88±4% and 87±5% and MMP-12 activity by 85±3% and 86±4% after oral and intrathecal delivery, respectively (FIG. 2F).

Moreover, in situ zymography of DC-injured spinal cord sections (Ahmed Z, Dent R G, Leadbeater W E, Smith C, Berry M, Logan A. Matrix metalloproteases: degradation of the inhibitory environment of the transected optic nerve and the scar by regenerating axons. Mol Cell Neurosci 2005; 28: 64-78) confirmed that DC injury-induced gelatinase activity (green; arrowheads) in the spinal cord adjacent to the lesion site (*) was completely suppressed by oral and intrathecally-administered AZD1236 (FIG. 2G). Taken together, these data demonstrate that AZD1236, delivered either oral or intrathecally, can significantly suppress DC injury-induced activity of MMP-9 and MMP-12.

Treatment with AZD1236 Reduces DC Injury-Induced Water Content (Oedema)

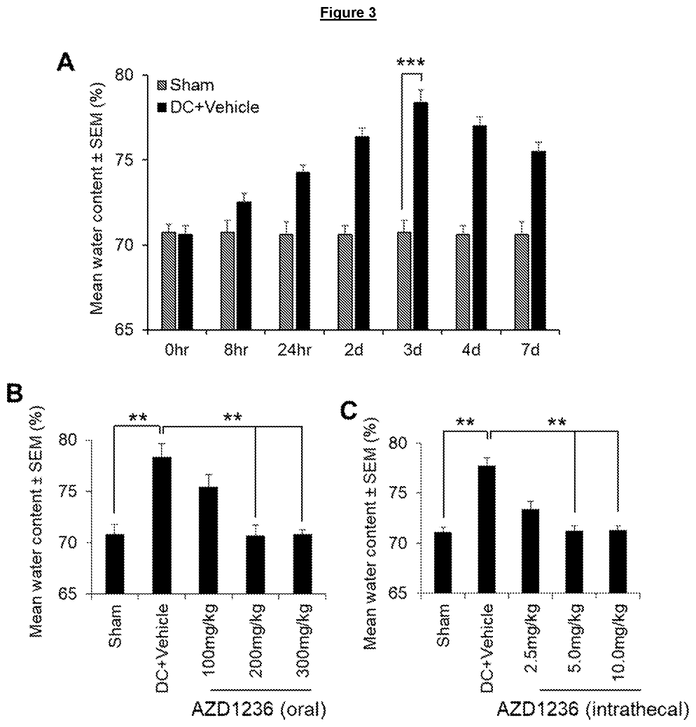

It was investigated if suppressing the early rise in MMP-9 and MMP-12 activity could reduce DC injury-induced water accumulation in the spinal cord. DC injury caused a significant increase in mean spinal cord water content from 71.0±1.0 to 78.4±1.5%, peaked at 3 days and declined thereafter (FIG. 3A). Oral delivery of clinically relevant doses of AZD1236, twice daily, caused a dose-dependent decrease in spinal cord water, reducing to sham-treated levels with 200 mg/kg and 300 mg/kg (FIG. 3B). Intrathecal delivery of AZD1236 also showed a similar dose-dependent decrease in spinal cord water content, although the doses required to elicit the same reduction as oral delivery required 1/40^{th} of the dose (FIG. 3C). Accordingly, DC injury-induced increase in spinal cord water content was reduced to sham-treated levels with 5.0 and 10.0 mg/kg AZD1236 (FIG. 3C). These results demonstrate that AZD1236 suppresses DC injury-induced oedema by both oral and intrathecal delivery, with intrathecal delivery requiring only 1/40^{th} of the oral dose.

It was then determined if attenuation of SCI-induced oedema is dependent on specific inhibition of MMP-9 or MMP-12. Using tool inhibitors (MMP-9 Inhibitor I and MMP408), either singly or in combination, it was shown that combined inhibition of MMP-9 and MMP-12 was required to fully halt SCI-induced oedema and that individual inhibition of MMP-9 or MMP-12 gave suboptimal therapeutic benefits (FIG. 3D). Taken together, these results show that AZD1236 inhibits SCI-induced oedema and that specific inhibition of both MMP-9 and MMP-12 is required to effectively halt SCI-induced oedema.

### CNS Edema is More Effectively Ablated by AZD1236 Compared to Other MMP Inhibitors and is Dependent on Specific Inhibition Both MMP-9 and MMP-12

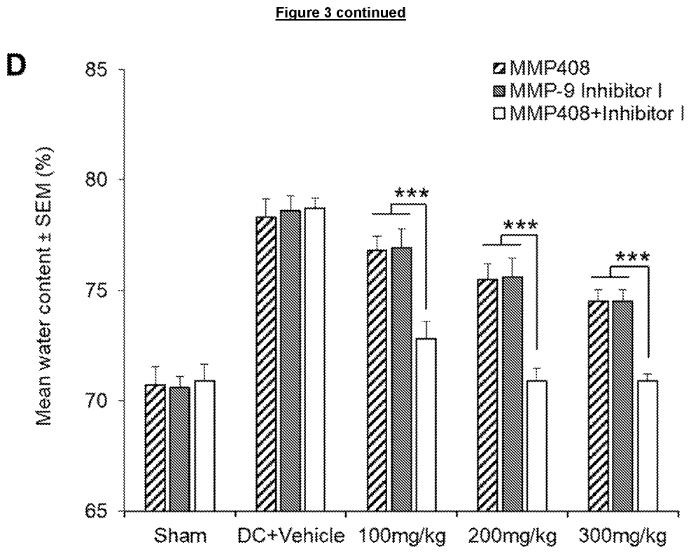

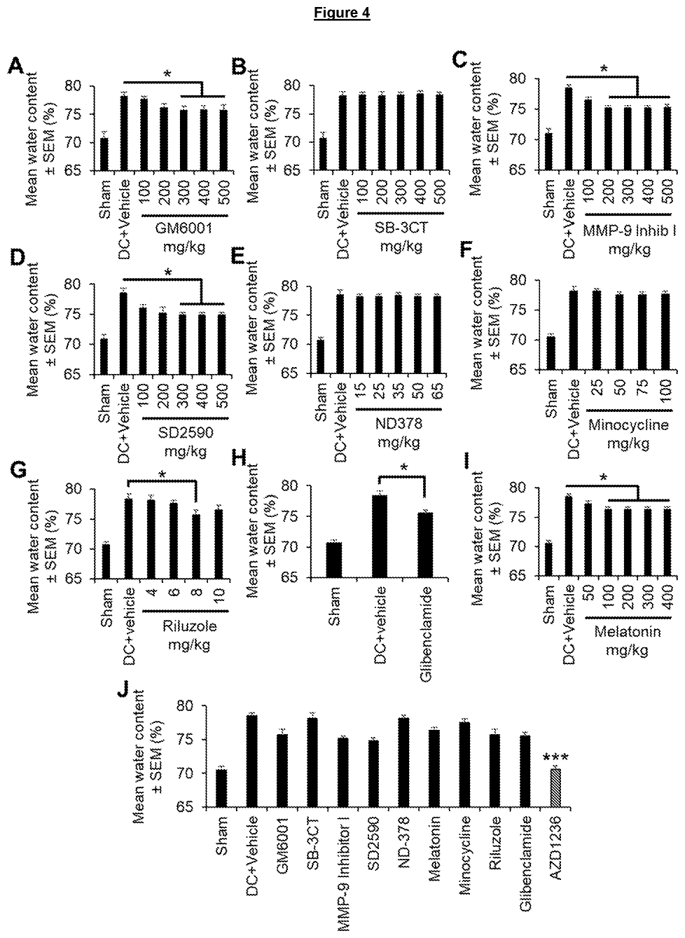

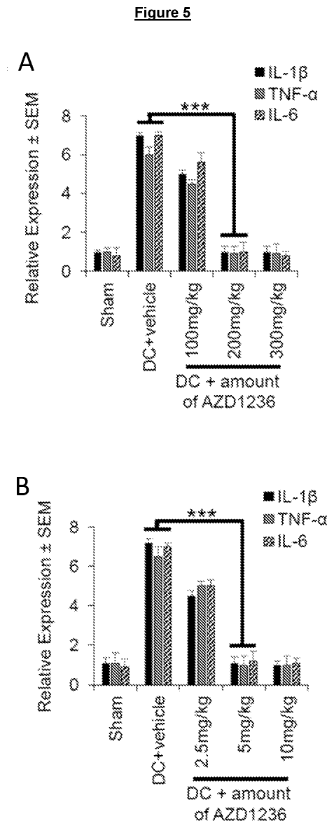

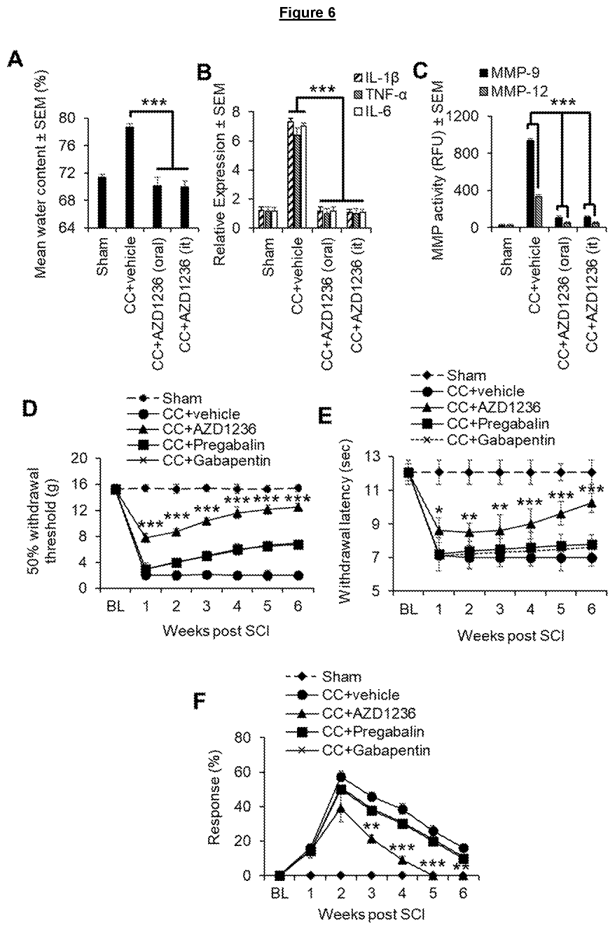

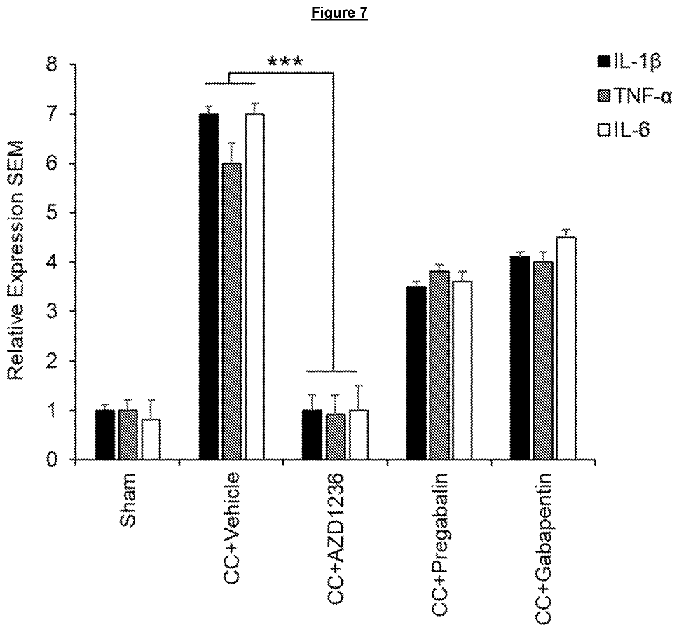

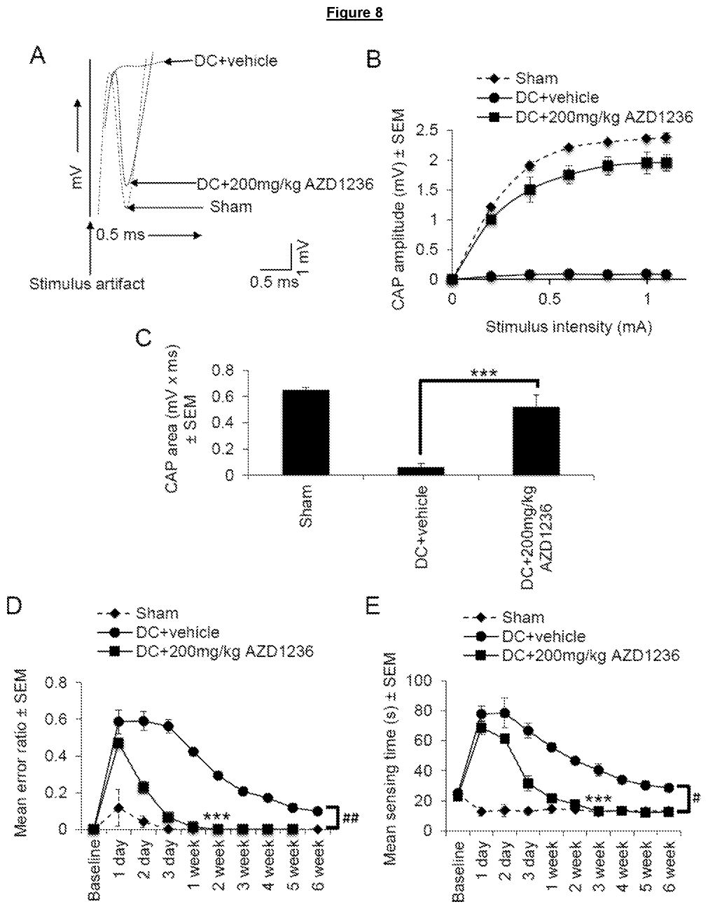

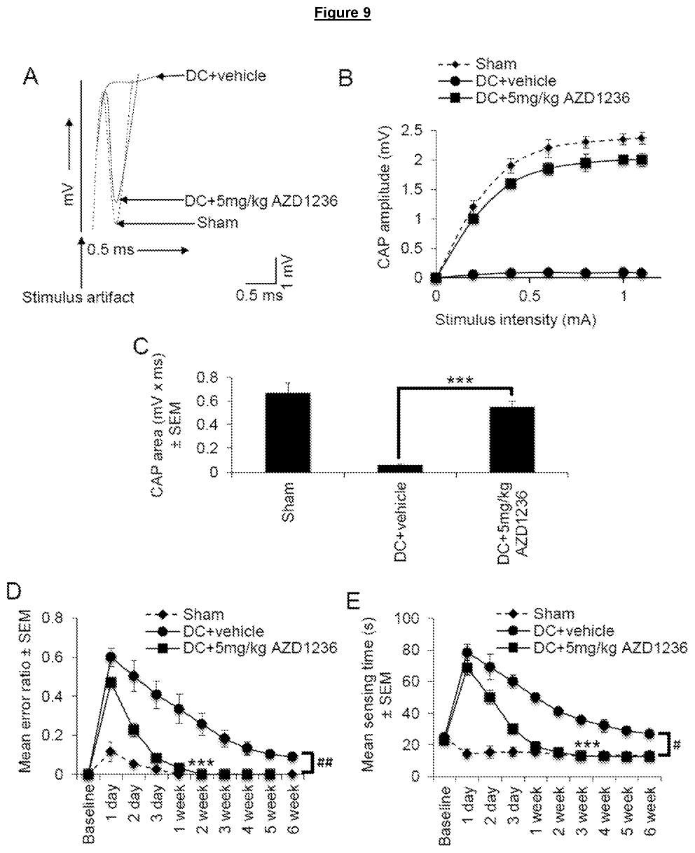

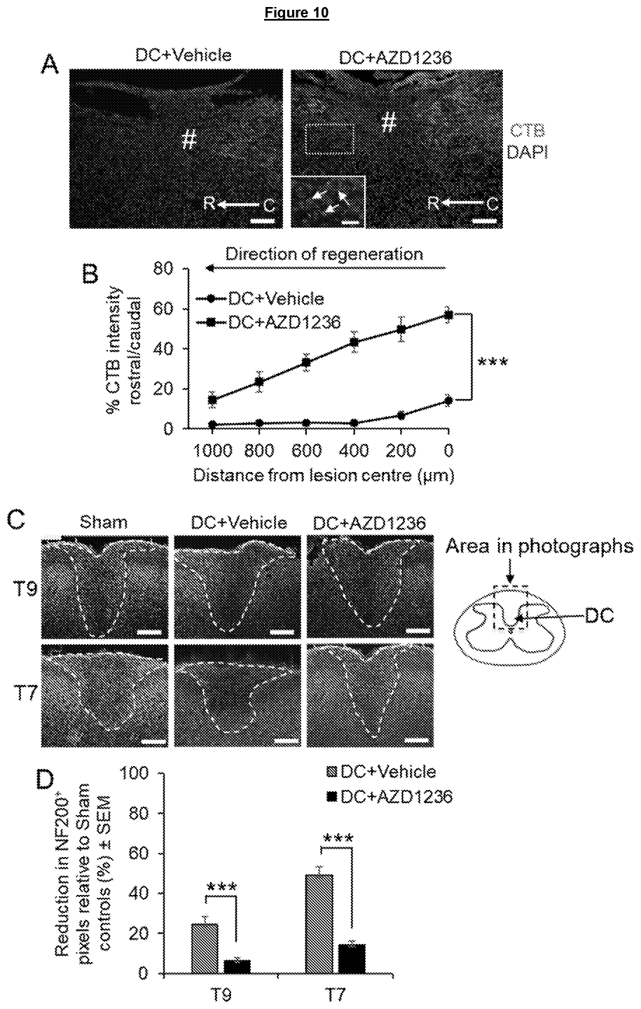

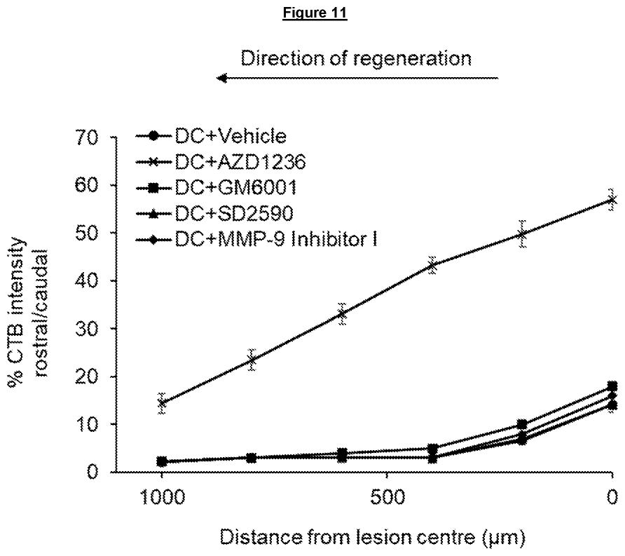

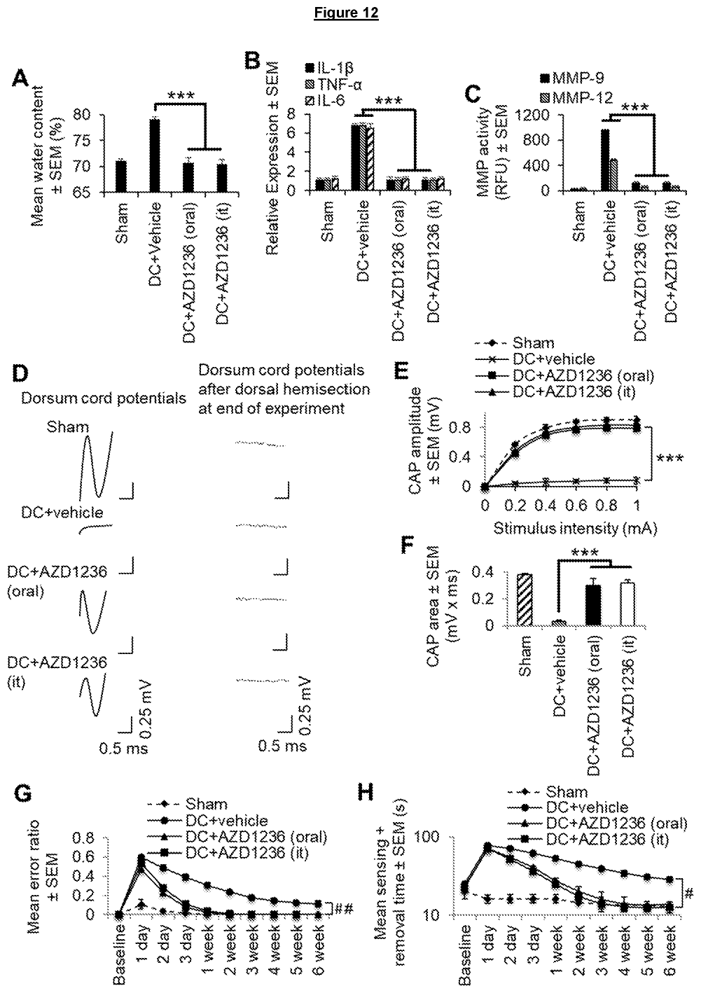

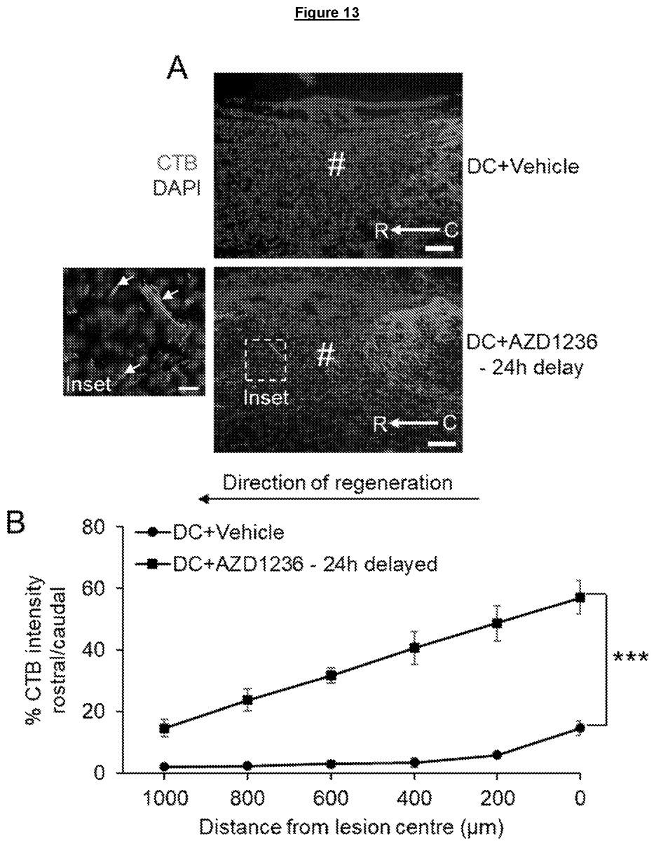

We compared oral delivery of AZD1236 with other published clinical and tool MMP inhibitors (indicated doses and routes—Table 1). Tool inhibitors included GM6001 (IC50=200 μM) (FIG. 4A), SB-CT (IC50=400 nM) (FIG. 4B), MMP-9 Inhibitor I (IC50=5 nM) (FIG. 4C), SD2590 (IC50=0.18 nM) (FIG. 4D) and ND378 (FIG. 4E).

GM6001 is a broad spectrum MMP inhibitor also known as Galardin or llomastat and has the chemical name (2R)—N⁴-Hydroxy-N¹-[(1S)-1-(1H-indol-3-ylmethyl)-2-(methylamino)-2-oxoethyl]-2-(2-methylpropyl)butanediamide. It is commercially available, for example from Tocris.

SB-3CT is a selective MMP-2 inhibitor and has the chemical name 2-[[(4-Phenoxyphenyl)sulfonyl]methyl]thiirane. It is commercially available, for example from Tocris.

SD2590 is a potent MMP inhibitor and has the chemical name N-Hydroxy-1-(2-methoxyethyl)-4-[4-[4-(trifluoromethoxy)phenoxy]phenyl]sulfonyl]-4-piperidinecarboxamide hydrochloride. It is commercially available, for example from Tocris.

Inhibitor I is an MMP-9 inhibitor (Ref. CAS 1177749-58-4) and is commercially available, for example from Sigma-Aldrich.

MMP408 is an MMP-12 inhibitor (Ref. CAS 1258003-93-8) and is commercially available, for example from Sigma-Aldrich.

Clinical grade experimental therapeutics which reportedly inhibit MMPs included Minocycline (IC50=272 μM) (FIG. 4F), Riluzole (FIG. 4G), Glibenclamide (FIG. 4H) and melatonin (FIG. 4I). Our data showed that, although melatonin, GM6001, MMP-9 Inhibitor I, SD2590, Riluzole and Glibenclamide significantly reduced spinal cord water content when compared to DC+vehicle-treated rats (P=0.05-0.01), AZD1236 was far more effective than any of these compounds (P=0.0001, AZD1236 vs melatonin, GM6001, MMP-9 Inhibitor I, SD2590, Riluzole and Glibenclamide) (FIG. 4J).

AZD1236 attenuates proinflammatory pain markers and behavioural measures of pain Proinflammatory pain markers IL-1β, TNF-α and IL-6 were all significantly upregulated by 6-7-fold at 3d after DC injury (FIGS. 5A and B). However, 200 mg/kg and 5 mg/kg of AZD1236 by either oral or intrathecal delivery, respectively, attenuated the injury-induced rise of these pain markers and reduced them to sham-treated levels (FIGS. 5A and B). These results demonstrated that AZD1236 suppresses proinflammatory pain markers after SCI.

The DC model is a moderate severity model of SCI and although sensor and locomotor deficits can be discriminated, it is unsuitable to detect the effects of AZD1236 on tactile, thermal and cold allodynia. However, the clip compression (CC) model is a severe injury and can be used to assess pain in these animals (Rivlin AS, Tator CH. Effect of duration of acute spinal cord compression in a new acute cord injury model in the rat. Surg Neurol 1978; 10(1): 38-43; Esposito E, Paterniti I, Mazzon E, Genovese T, Galuppo M, Meli R, et aL. MK801 attenuates secondary injury in a mouse experimental compression model of spinal cord trauma. BMC Neurosci 2011; 12: 31). The CC model of SCI was therefore employed to investigate if inhibition of MMP-9 and MMP-12 using AZD1236 attenuates tactile, thermal and cold allodynia. We first confirmed that treatment of the CC mouse with same doses of AZD1236 as the DC injury model, either by oral or intrathecal in the clip compression model caused a similar attenuation of spinal cord water content, expression of IL-1β, TNF-α and IL-6 and MMP-9/MMP-12 activity (FIG. 6A-C) to that seen in the DC model. Suppression of proinflammatory markers, spinal cord water content and MMP-9/MMP-12 activity by AZD1236 all significantly improved (to >80% of sham-treated controls) measures of mechanical (FIG. 6D), thermal (FIG. 6E) and cold (FIG. 6F) allodynia. In addition, improvements in mechanical, thermal and cold allodynia were significantly better in AZD1236 treated animals than those treated with currently approved neuropathic pain medications, pregabalin and gabapentin (FIG. 6D-F). These improvements correlated with the superior ability of AZD1236 to suppress proinflammatory pain markers, IL-1β, TNF-α and IL-6, compared to pregabalin and gabapentin (FIG. 7). These results suggest that AZD1236 may also be useful in suppressing SCI-induced neuropathic pain and may even be more effective than currently used pain medications.

### AZD1236 Preserves CAP Amplitudes and Improves Locomotor and Sensory Function

Compound action potentials (CAPs) across the spinal cord lesion site were measured by electrophysiology with and without AZD1236 treatment to determine if suppression of spinal cord water content by AZD1236 improved functional outcomes after DC injury.

Superimposed representative CAP traces from Spike 2 software-processed Sham control, DC+vehicle and DC+200 mg/kg AZD1236 showed that in DC+vehicle groups the negative CAP wave was ablated compared to Sham-treated controls (FIG. 8A). However, treatment with oral AZD1236 restored a significant CAP wave (FIG. 8A). Dorsal hemisection of the spinal cord at the end of the experiment abolished the CAP trace in all animals and confirmed that the experiment was technically successful. The mean CAP amplitude was also ablated in DC+vehicle-treated groups whilst significantly larger CAP amplitudes were observed after AZD1236 treatment at all stimulation intensities (FIG. 8B). Likewise, the CAP area in Sham-treated groups (0.65±0.02 mV×ms) was reduced to 0.06±0.02 mV×ms in DC+vehicle-treated groups but was restored to 80% of that observed in Sham-treated controls (0.52±0.09 mV×ms) (FIG. 8C). These improvements in the electrophysiological properties across the DC lesion site translated into significant improvements (85% better compared to sham-treated animals) in both locomotor and sensory function. For example, the mean error ratio for the ladder crossing test remained between 0.15 and 0 throughout the 6-week time period (FIG. 8D). After DC injury, the mean error ratio increased to 0.59±0.06 and a significant deficit (P<0.0012, generalized linear mixed model) remained throughout the 6-week time period.

However, treatment with AZD1236 improved the mean error ratio at all time points such that by 2 weeks after injury, AZD1236 treated animals had improved significantly (P<0.0001, independent sample t-test) and were indistinguishable from sham-treated animals (FIG. 8D). The mean tape sensing and removal time also increased to 77.8±5.0 s in DC+vehicle-treated animals (FIG. 8E). AZD1236 treatment significantly reduced the mean tape sensing and removal time, showing significant improvements compared to DC+vehicle-treated groups (P<0.0001, independent sample t-test) such that by 3 weeks, animals were indistinguishable from sham-treated groups (FIG. 8E).

Intrathecal delivery of the lowest effective dose of AZD1236 also caused similar significant improvements in CAP waves, CAP amplitudes, CAP areas, mean error ratios and mean tape sensing and removal times compared to oral AZD1236 delivery (FIG. 9A-E).

These results demonstrate that inhibition of MMP-9 and MMP-12 using AZD1236 significantly improved electrophysiological, locomotor and sensory function. These results also demonstrate that oral and intrathecal delivery of AZD1236 caused similar beneficial improvements in electrophysiological, locomotor and sensory function after DC injury.

Inhibition of MMP-9 and MMP-12 with AZD1236 Promotes DC Axon Regeneration and Sparing of Axons Above and Below the Lesion Site

DC axon regeneration/sprouting was then investigated using the retrograde tracer, Cholera Toxin B (CTB) to label regenerating fibres at the lesion site. In DC+Vehicle-treated mice no CTB labelled axons (red) were observed beyond the lesion site (#) (FIG. 10A). However, in DC+AZD1236-treated mice, CTB⁺ axons were observed regenerating through lesion site (#) and extending into the rostral cord (FIG. 10A and inset, showing high power view of axons in the rostral cord). Quantification of CTB⁺ labeling intensity showed significantly enhanced percentage of CTB labelled axons in the DC+AZD1236-treated groups compared to DC+Vehicle-treated groups (P=0.0001, ANOVA) (FIG. 9B) extending at least 1000 μm from the lesion centre.

Immunostaining for NF200⁺ spared fibres in cross-sections of the spinal cord at T9 (above the lesion) and T7 (below the lesion) (FIG. 10C) and subsequent quantification (FIG. 10D) showed that AZD1236 significantly preserved axon fibres at both above (T9) and below (T7) the lesion site. These results demonstrate that oral AZD1236 not only promoted DC axon regeneration across the lesion site but also enhanced their preservation both above and below the lesion site.

AZD1236 Promotes Significantly More DC Axon Regeneration than Other MMP Inhibitors

Quantification of the number of CTB⁺ DC axons regenerating after treatment with AZD1236 were also compared with other MMP inhibitors, such as GM6001, SDF2590 and MMP-9 Inhibitor I (best performing MMP inhibitors at reducing oedema after DC injury). We found that AZD1236 was far superior at promoting DC axon regeneration after injury, with all other MMP inhibitors having only a marginal effect on axon regeneration (FIG. 11).

Delayed Inhibition of MMP-9 and MMP-12 is Also Beneficial after DC Injury

Since it may take several hours to establish the exact clinical diagnosis and treatment regimen for an SCI patient, a clinically-relevant, 24 h time delay prior to dosing animals with AZD1236 was assessed. Even with this 24-h delay, optimal doses of AZD1236 were equally as effective as immediate delivery, in suppressing SCI-induced water content (FIG. 12A), proinflammatory cytokines (FIG. 12B) and MMP-9 and MMP-12 activity (FIG. 12C) as well as improving dorsum cord potentials (FIG. 11D), CAP amplitudes (FIG. 12E), CAP areas (FIG. 12F), locomotor (FIG. 12G) and sensory function (FIG. 12H). Moreover, 24 h delayed treatment with AZD1236 promoted the same amount of CTB⁺ fibres regenerating through the lesion site (#) and entering into the rostral cord (FIGS. 13A and B). These results demonstrate the significant potential for clinical utility of AZD1236 for SCI, given that a clinically-relevant time delay to treatment with AZD1236, was just as effective as immediate treatment.

### Inhibition of MMP-9 and MMP-12 by AZD1236 (Oral Dose) Attenuates BSCB Breakdown, Scarring, Activation of Microglia and Infiltration of Macrophages at the Lesion Site

The blood-spinal cord barrier (BSCB) is functionally equivalent to the blood brain barrier (BBB) and responsible for providing a specialized microenvironment for the extracellular constituents of the spinal cord (Bartanusz V, Jezova D, Alajajian B, Digicaylioglu M. The blood-spinal cord barrier: morphology and clinical implications. Ann Neurol 2011; 70(2): 194-206). Damage to the BSCB results after SCI, causing progressive hemorrhage that leads to secondary injury mechanisms and permanent neurological deficits (Tran A P, Warren P M, Silver J. The Biology of Regeneration Failure and Success After Spinal Cord Injury. Physiol Rev 2018; 98(2): 881-917). The integrity of the BSCB was assessed using albumin immunoreactivity in the spinal cord at 3 days (or 4 days for 24 h delayed group) after injury and treatment, as a surrogate marker of BSCB disruption.

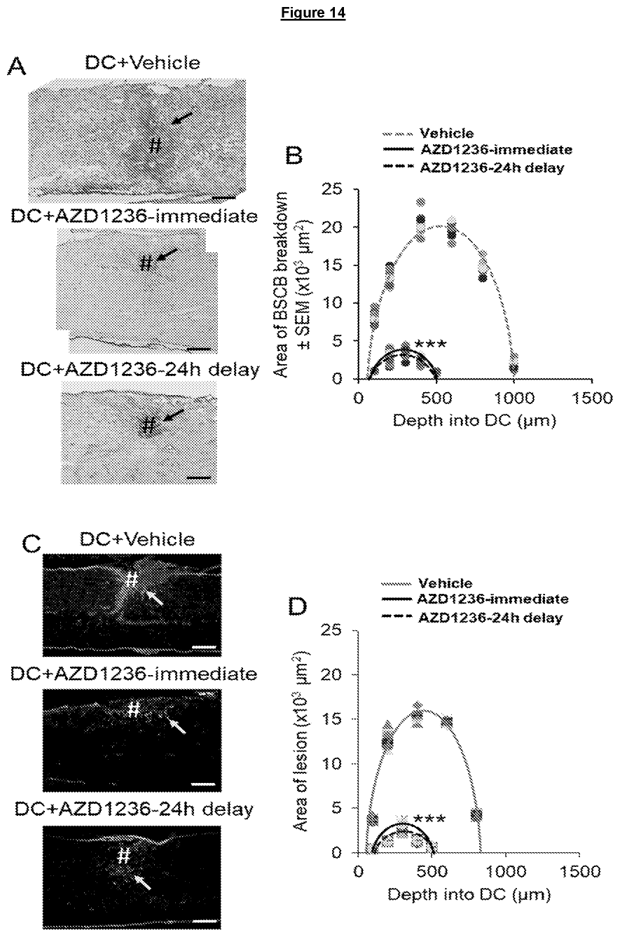

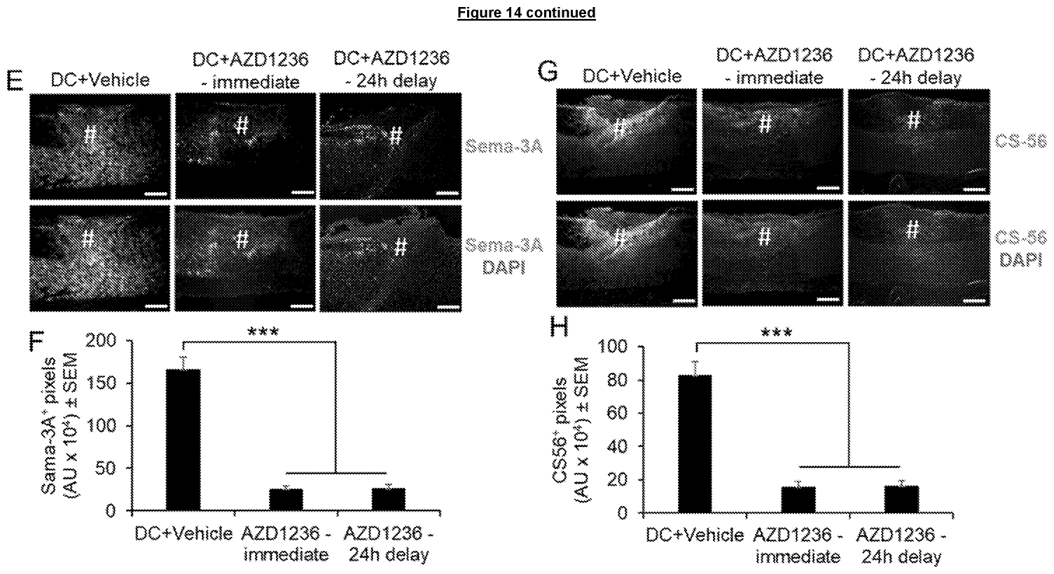

Significant albumin immunoreactivity (arrow) was observed throughout the lesion site (#) and surrounding spinal cord parenchyma in sections at 3 days post-injury; showed that in DC+vehicle-treated groups, significant BSCB breakdown occurred acutely at all depths throughout the spinal cord at 3d after injury (FIGS. 14A and B). However, treatment with oral AZD1236, both immediately after injury and with a 24 h delay to treatment, significantly suppressed this area of BSCB breakdown (arrow) by >75% compared to DC+vehicle-treated groups (P<0.0001, ANOVA) and by >73% in groups that received 24 h delayed AZD1236 treatment (FIGS. 14A and 14B), demonstrating protection against BSCB breakdown.

Likewise, laminin immunoreactivity to demarcate scar tissue extracellular matrix at 4 weeks after DC injury, showed a large area of staining (arrow) at the lesion site (#) indicative of significant scarring present in DC+vehicle-treated animals (FIGS. 14C and D). Only low levels of laminin immunoreactivity were present in both groups receiving AZD1236 immediately after injury (attenuated>80%, P=0.0001, ANOVA) or 24 hour delayed treated (attenuated by >77%, P=0.00011, ANOVA) (FIGS. 14C and D).

These results suggest that AZD1236 suppresses both BSCB breakdown (acute) and lesion site scarring when dosed immediately or 24 hours after injury.

AZD1236 treatment, whether immediate or delayed by 24 h after injury, also significantly suppressed Sema-3A ((FIGS. 14E and F) (attenuated by >84%, P=0.0001, ANOVA) and CS-56 ((FIGS. 14G and H) (attenuated by >81%, P<0.0001, ANOVA)) immunoreactivity at the lesion site at 7 days (or at 8 days for the 24 h delayed group) after injury and treatment, indicating protection against the deposition of scar-related molecules in the injury site. Furthermore, AZD1236 treatment, whether immediate or delayed by 24 h also attenuated CD11b (FIGS. 14I and J; Inset i=high power view of boxed region to show microglial activation), CD68 (FIGS. 14I and K; inset ii=high power view of boxed region to show macrophage activation/infiltration) and GFAP⁺ (FIGS. 14I and L) immunoreactivity at the lesion site, which were all significantly reduced (P=0.0001, ANOVA) when quantified by image analysis (FIG. 14J-L).

These results demonstrate that AZD1236 treatment, whether immediate or delayed by 24 h, equally and robustly suppress SCI-induced BSCB breakdown, scarring at the lesion site, infiltration of macrophages and activation of microglia and astrocytes at the lesion site.

Inhibition of MMP-9 and MMP-12 with AZD1236 Ablates Common CSF Biomarkers of SCI

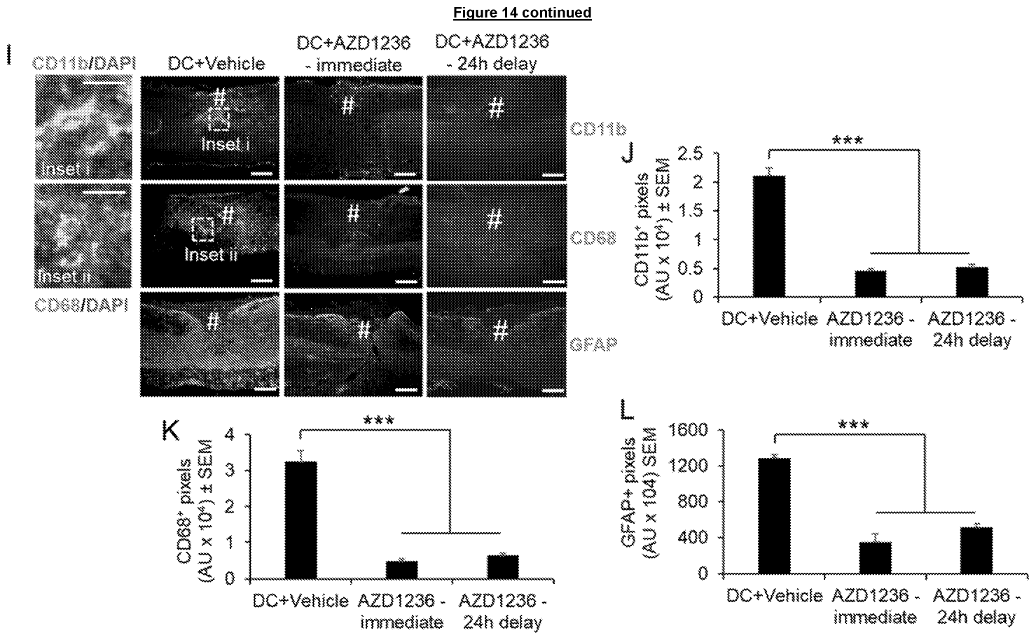

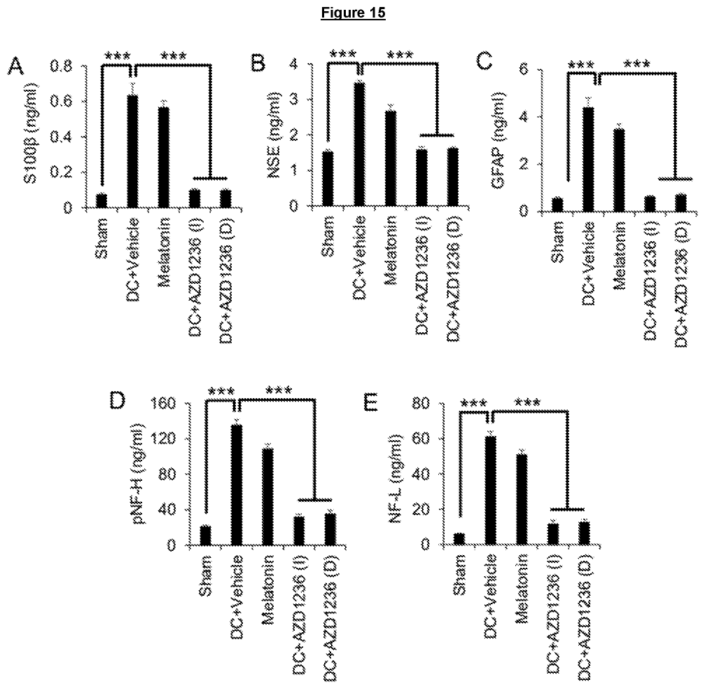

Biomarkers in the CSF and serum are increasingly being used to stratify SCI injury, in terms of severity and the potential to recover after injury. Some of the most common reported biomarkers after SCI were analysed, including S100β, neuron-specific enolase (NSE), glial fibrillary acidic protein (GFAP), phosphorylated neurofilament heavy chain (pNF-H) and neurofilament light chain (NF-L) and compared their levels after AZD1236 treatment. The levels of S1003β, NSE, GFAP, pNF-H and NF-L were all significantly elevated at 3 days after injury (FIG. 15A-E). Treatment with melatonin marginally reduced the levels of these biomarkers in CSF whilst AZD1236, either immediately or delayed by 24 h significantly attenuated all of these biomarkers to near sham-control levels at 3 d after injury. These results suggest that AZD1236 also helps to suppress common biomarkers of SCI, which could be used clinically to monitor SCI progression.

AZD1236 Suppresses LPS-Induced Cytokine Production by Microglia and but does not Affect Macrophage Migration In Vitro

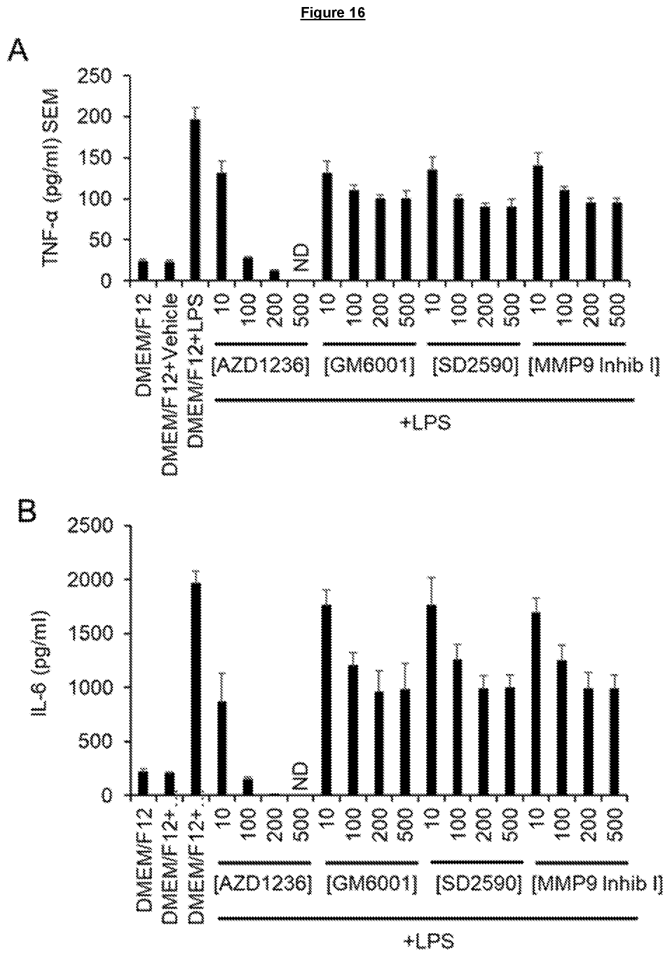

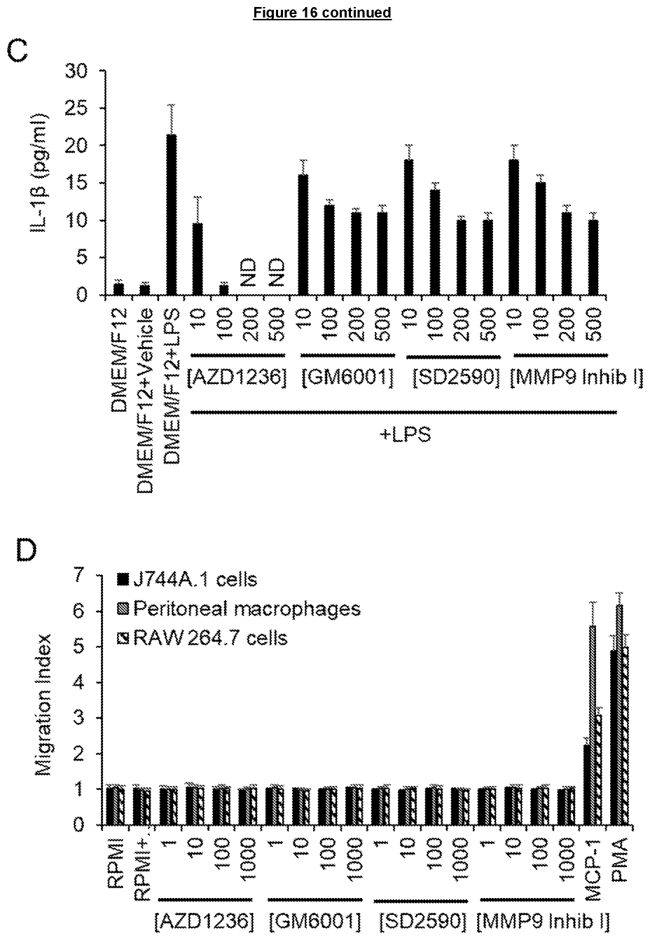

To determine the effect of AZD1236 on microglial activation and macrophage migration, primary adult mouse brain microglia were isolated and subjected them to LPS activation, with and without AZD1236 treatment and then performed ELISA for TNF-α, IL-1β and IL-6. 10 ng/ml of LPS, stimulated the production of 196±15, 21±4 and 1964±117 pg/ml of TNF-α, IL-1β and IL-6, respectively (FIG. 16A-C). However, LPS activation together with increasing concentrations of AZD1236 caused a dose-dependent decrease in these cytokines, with 100 ng/ml of AZD1236 returning their concentrations to DMEM/F12-medium control levels (FIG. 16A-C). Increasing the concentration of AZD1236 to 500 ng/ml completely suppressed the production of these cytokines, despite the presence of LPS to activate microglia (FIG. 16A-C). In contrast, other MMP inhibitors, such as GM6001, SD2590 and MMP 9 Inhibitor I had marginal effects on suppression of these cytokines (FIG. 16A-C).

AZD1236 and other MMP inhibitors had no effect on chemotaxis in primary macrophages or J744A.1 and RAW 264.7 macrophage cell lines, since the migration index remained at baseline control levels (FIG. 16D). Meanwhile, the positive controls, MCP-1 and PMA, both stimulated significant chemotaxis in primary peritoneal macrophages and both macrophage cell lines (FIG. 16D).

These results demonstrate that AZD1236 significantly suppresses microglial activation and thus secretion of cytokines, such as TNF-α, IL-1β and IL-6, but does not affect migration of macrophages.

### Inhibition of MMP-9 and MMP-12 is Also Effective in Rat Models of SCI

It was investigated if suppression of MMP-9 and MMP-12 could also attenuate edema and improve functional recovery in a rat DC injury model, which better recapitulates human SCI pathophysiology (Surey S, Berry M, Logan A, Bicknell R, Ahmed Z. Differential cavitation, angiogenesis and wound-healing responses in injured mouse and rat spinal cords. Neuroscience 2014; 275: 62-80). For example, like humans, rats also form fluid-filled cysts that enlarge over time after DC injury; a response that further damages spinal cord tissues and disrupts axons. In addition, the fluid-filled cysts are surrounded by scar tissue that presents an additional barrier to regenerating/sprouting axons.

We showed that the same profile of MMP-9 (FIG. 17A) and MMP-12 (FIG. 17B) mRNA expression was present after DC injury in the rat when compared to mice. Since AZD1236 is not active against rat MMP-9/-12, AZD3342—an MMP inhibitor with similar selectivity to AZD1236 that is active in the rat (IC₅₀=117 nM and 35 nM against rat MMP-9 and -12, respectively)—was used to demonstrate that SCI-induced edema (FIG. 17C), MMP-9 and MMP-12 activity (FIG. 17D), and proinflammatory pain related cytokines (FIG. 17E) could also be suppressed in a rat DC injury model. Treatment with AZD3342 significantly improved electrophysiological CAP traces (FIG. 17F) and locomotor (FIG. 17G) and sensory function (FIG. 17H), similar to that observed with AZD1236 in the mouse SCI model. All of these changes in edema, proinflammatory pain markers, CAP traces, locomotor and sensory function were markedly better than melatonin treatment (used as a positive control). These results demonstrate that inhibition of MMP-9 and MMP-12 is also effective against SCI-induced edema, neuropathic pain and protects against functional loss in rat models of SCI.

Normal Injury Site MMP-9 and MMP-12 Activity Levels Return by 5 d after Withdrawal of AZD1236

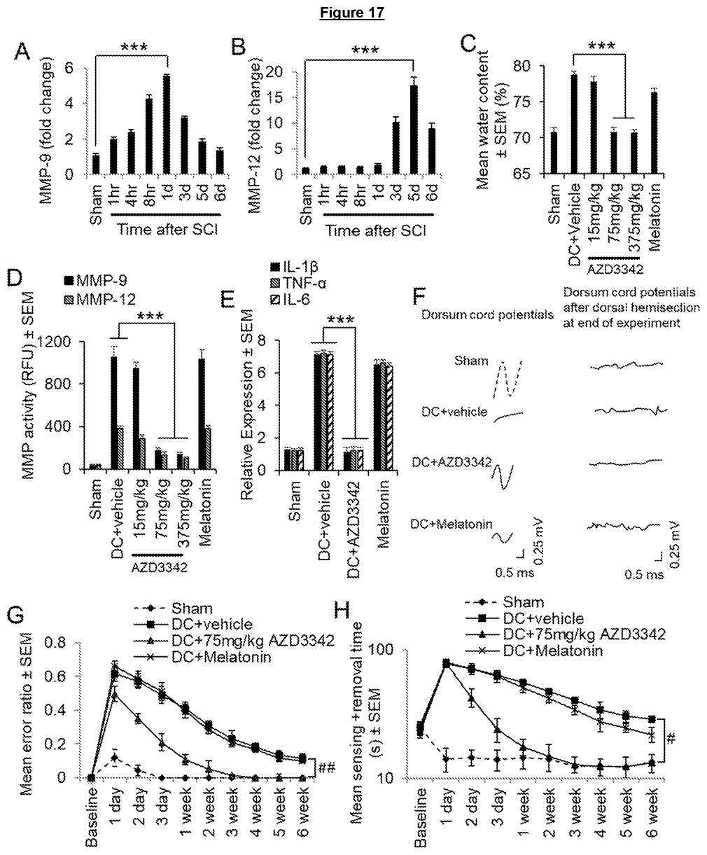

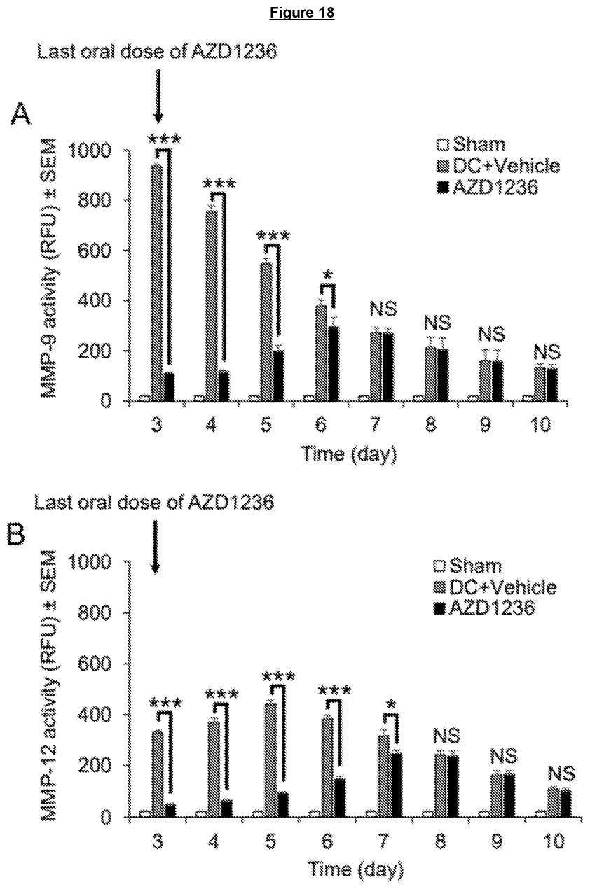

It was investigated the time it takes for MMP-9 and MMP-12 to return to normal SCI-induced activity levels in the injury site, once oral AZD1236 is withdrawn after 3 d. The results demonstrate that MMP-9 took 4 d whilst MMP-12 took 5 d to return to activity levels observed after DC+Vehicle injury groups (FIGS. 18A and B). These results demonstrate that normal MMP-9 and MMP-12 activity levels return, 4-5 days after the last dose of AZD1236, well before the wound healing phase in the CNS.

AZD1236 Treatment does not Affect MMP-2 Activity

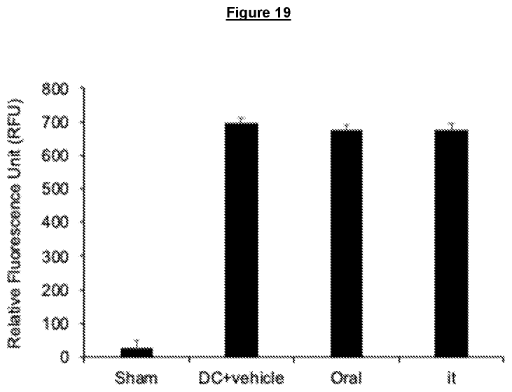

It was investigated if oral and intrathecal delivery of the effective dose of AZD1236 inadvertently affected MMP-2 activity. However, MMP-12 activity assays demonstrated no difference in DC injury-induced rise in MMP-2 activity after treatment with AZD1236, suggesting no off-target effects on MMP-2 activity by AZD1236 at the doses used (FIG. 19).

### Example 2

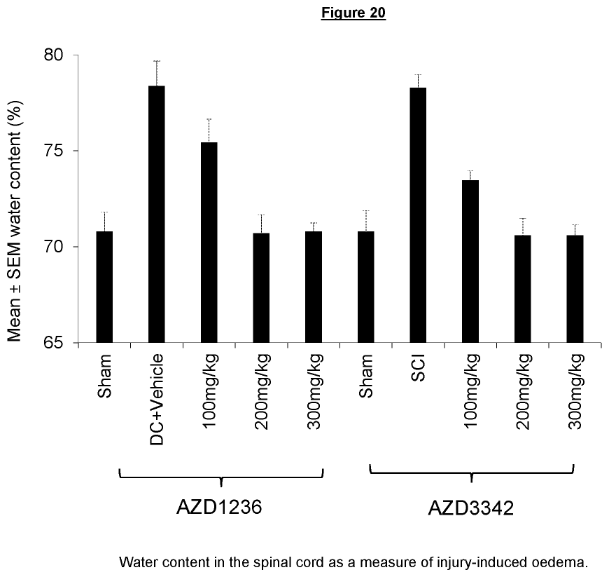

Using similar techniques to those described in Example 1, the water content in the spinal cord as a measure of injury-induced oedema was determined for both AZD1236 & AZD3342 (see FIG. 20).

### Example 3

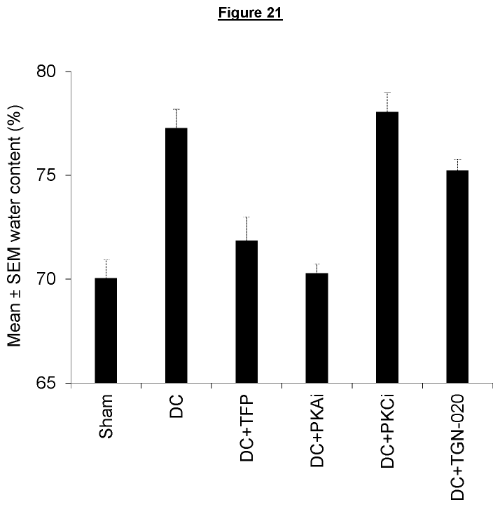

Using similar techniques to those described in Example 1, the water content in the spinal cord as a measure of injury-induced oedema was determined for various agents and aquaporin-4 inhibitors, namely TFP, PKAi, PKCi and TGN-020—see FIG. 21.

TFP=trifluoperazine is also known as Stelazine, Eskazinyl, Eskazine or Jatroneural and is commercially available.

PKAi is a protein kinase A inhibitor and PKCi is a protein kinase C inhibitor.

TGN-020 is an aquaporin 4 (AQP4) channel blocker and has the chemical name N-1,3,4-Thiadiazol-2-yl-3-pyridinecarboxamide. It is commercially available, for example from Tocris.

1. A compound or combination of compounds for use in treating spinal cord injury (SCI) or related injury to neurological tissue or for use in treating the secondary effects associated with SCI or related injury to neurological tissue, comprising selectively inhibiting the activity or expression of both matrix metalloproteinase MMP-9 (gelatinase-B) and metalloelastase MMP-12 after such SCI or related injury to neurological tissue.

2. A compound or combination of compounds for use according to claim 1 in treating the secondary effects associated with SCI or related injury to neurological tissue, comprising selectively inhibiting the activity or expression of both matrix metalloproteinase MMP-9 (gelatinase-B) and metalloelastase MMP-12 after such SCI or related injury to neurological tissue.

3. A compound or combination of compounds for use according to claim 2 wherein the secondary effect treated is SCI-induced oedema or neuropathic pain (NP).

4. A compound or combination of compounds for use according to claim 3 wherein the SCI-induced oedema is completely suppressed or is suppressed by 25%-50%, for example by 30%, following treatment.

5. A compound or combination of compounds for use according to claim 1 or 2 wherein the related injury to neurological tissue is traumatic brain injury or stroke.

6. A compound or combination of compounds for use according to any one of claims 1 to 5 wherein a single selective MMP-9 and MMP-12 inhibitor compound or a combination of a selective MMP-9 compound and a separate selective MMP-12 inhibitor compound is used.

7. A compound for use according to any one of claims 1 to 5 wherein a single selective MMP-9 and MMP-12 inhibitor compound is used.

8. A compound for use according to claim 6 or 7 wherein the single selective MMP-9 and MMP-12 inhibitor compound has an IC₅₀ against both MMP-9 and MMP-12 in the range of 1 nM to 100 nM.

9. A compound for use according to claim 6 or 7 wherein the single selective MMP-9 and MMP-12 inhibitor compound has an IC₅₀ of greater than 100 nM against MMP-2.

10. A compound for use according to claim 8 or 9 wherein the single selective MMP-9 and MMP-12 inhibitor compound has an IC₅₀ against both MMP-9 and MMP-12 in the range of 1 nM to 100 nM and an IC₅₀ of greater than 200 nM against MMP-2.

11. A compound for use according to claim 6, 7 or 8 wherein the single selective MMP-9 and MMP-12 inhibitor compound is AZD1236 or AZD3342, or a pharmaceutically acceptable salt of these.

12. A compound for use according to claim 6, 7, 8 or 11 wherein a single selective MMP-9 and MMP-12 inhibitor compound is used and the single selective MMP-9 and MMP-12 inhibitor compound is AZD1236, or a pharmaceutically acceptable salt thereof.

13. A compound or combination of compounds for use according to any one of claims 1 to 12 wherein the activity or expression of MMP-9 and MMP-12 is inhibited by administering a single selective MMP-9 and MMP-12 inhibitor compound or a combination of a selective MMP-9 and a separate selective MMP-12 inhibitor compound for a short-duration following SCI or related injury to neurological tissue, for example over one, two or three days.

14. A compound or combination of compounds for use according to any one of claims 1 to 13 wherein the activity or expression of MMP-9 and MMP-12 is inhibited by intrathecal administration of a single selective MMP-9 and MMP-12 inhibitor or a combination of a selective MMP-9 and a separate selective MMP-12 inhibitor.

15. A compound or combination of compounds for use according to any one of claims 1 to 14 wherein the activity or expression of MMP-9 and MMP-12 is inhibited by intrathecal administration of a single selective MMP-9 and MMP-12 inhibitor or a combination of a selective MMP-9 and a separate selective MMP-12 inhibitor at 1/20^{th} to 1/60^{th} of the oral dose required, typically 1/40^{th}.

16. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for treating SCI-induced oedema.

17. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for treating neuropathic pain (NP).

18. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for reducting BSCB breakdown.

19. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for reducing blood-borne cells in the CNS.

20. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for reducing scarring at the SCI lesion site.

21. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for the prevention of scarring.

22. A compound for use according to any one of claims 6 to 15 wherein a single selective MMP-9 and MMP-12 inhibitor compound is used and the single selective compound is AZD1236, or a pharmaceutically acceptable salt thereof, and wherein AZD1236, or a pharmaceutically acceptable salt thereof, is used for promoting of axon regeneration.

23. A compound for use according to any one of claims 1 to 22 wherein the compound is AZD1236, or a pharmaceutically acceptable salt thereof, and wherein the compound or a pharmaceutically acceptable salt thereof is administered for a short-duration following SCI or related injury to neurological tissue, for example over one, two or three days.

24. A compound for use according to claim 23 wherein AZD1236, or a pharmaceutically acceptable salt thereof is orally dosed at 50 to 100 mg twice daily, such as about 75 mg twice daily.

25. A method for treating spinal cord injury (SCI) or related injury to neurological tissue, or for treating the secondary effects associated with SCI or related injury to neurological tissue, comprising selectively inhibiting the activity or expression of matrix metalloproteinases MMP-9 (gelatinase-B) and metalloelastase MMP-12 after such SCI or related injury to neurological tissue.

26. A method according to claim 25 for treating the secondary effects associated with spinal cord injury (SCI) or related injury to neurological tissue, comprising selectively inhibiting the activity or expression of matrix metalloproteinases MMP-9 (gelatinase-B) and metalloelastase MMP-12 after such SCI or related injury to neurological tissue.
